SEQUENCE LISTING (34 sequences)

SEQ ID NO: 1 - DNA, 14 bp, Artificial sequence
  Synthetic primer
tttcaccgctacac

SEQ ID NO: 2 - DNA, 15 bp, Artificial sequence
  Synthetic primer
atatggaggaacacc

SEQ ID NO: 3 - DNA, 15 bp, Artificial sequence
  Synthetic primer
atatggaggaacacc

SEQ ID NO: 4 - DNA, 14 bp, Artificial sequence
  Synthetic primer
tcaagtcatcatgg

SEQ ID NO: 5 - DNA, 14 bp, Artificial sequence
  Synthetic primer
tcaaatcatcatgc

SEQ ID NO: 6 - DNA, 14 bp, Artificial sequence
  Synthetic primer
caagtcatcatggc

SEQ ID NO: 7 - DNA, 14 bp, Artificial sequence
  Synthetic primer
caaatcatcatgcc

SEQ ID NO: 8 - DNA, 19 bp, Artificial sequence
  Synthetic primer
ggaattactgggcgtaaag

SEQ ID NO: 9 - DNA, 18 bp, Artificial sequence
  Synthetic primer
tttattgggcgtaaagcg

SEQ ID NO: 10 - DNA, 18 bp, Artificial sequence
  Synthetic primer
ctaccagggtatctaatc

SEQ ID NO: 11 - DNA, 23 bp, Artificial sequence
  Synthetic primer
gtggactaccagggtatctaatc

SEQ ID NO: 12 - DNA, 20 bp, Artificial sequence
  Synthetic primer
gagcatgtggtttaattcga

SEQ ID NO: 13 - DNA, 18 bp, Artificial sequence
  Synthetic primer
agcatgtggtttaattcg

SEQ ID NO: 14 - DNA, 18 bp, Artificial sequence
  Synthetic primer
aacgcgaagaaccttacc

SEQ ID NO: 15 - DNA, 18 bp, Artificial sequence
  Synthetic primer
ggcatgatgatttgacgt

SEQ ID NO: 16 - DNA, 19 bp, Artificial sequence
  Synthetic primer
tgtagcccaggtcataagg

SEQ ID NO: 17 - DNA, 18 bp, Artificial sequence
  Synthetic primer
tgtagcccaggtcataag

SEQ ID NO: 18 - DNA, 20 bp, Artificial sequence
  Synthetic primer
cattgtagcacgtgtgtagc

SEQ ID NO: 19 - DNA, 14 bp, Artificial sequence
  Synthetic probe
gtgtagcggtgaaa

SEQ ID NO: 20 - DNA, 14 bp, Artificial sequence
  Synthetic peptide formula
gtgtancggtgaaa

SEQ ID NO: 21 - DNA, 14 bp, Artificial sequence
  Synthetic probe
gtgtagcggtgaaa

SEQ ID NO: 22 - DNA, 14 bp, Artificial sequence
  Synthetic peptide formula
gtgtagcggtgaaa

SEQ ID NO: 23 - DNA, 15 bp, Artificial sequence
  Synthetic probe
atatggaggaacacc

SEQ ID NO: 24 - DNA, 15 bp, Artificial sequence
  Synthetic peptide formula
nnnnnnangnannnc

SEQ ID NO: 25 - DNA, 15 bp, Artificial sequence
  Synthetic probe
atatggaggaacacc

SEQ ID NO: 26 - DNA, 15 bp, Artificial sequence
  Synthetic peptide formula
atatggaggaacacc

SEQ ID NO: 27 - DNA, 14 bp, Artificial sequence
  Synthetic probe
tcaagtcatcatgg

SEQ ID NO: 28 - DNA, 14 bp, Artificial sequence
  Synthetic peptide formula
tcaantcnncangg

SEQ ID NO: 29 - DNA, 14 bp, Artificial sequence
  Synthetic probe
tcaagtcatcatgg

SEQ ID NO: 30 - DNA, 14 bp, Artificial sequence
  Synthetic peptide formula
tcaaatcatcatgc

SEQ ID NO: 31 - DNA, 14 bp, Artificial sequence
  Synthetic probe
tcaaatcatcatgc

SEQ ID NO: 32 - DNA, 14 bp, Artificial sequence
  Synthetic peptide formula
tcaantcnncangg

SEQ ID NO: 33 - DNA, 14 bp, Artificial sequence
  Synthetic probe
tcaaatcatcatgc

SEQ ID NO: 34 - DNA, 14 bp, Artificial sequence
  Synthetic peptide formula
tcaaatcatcatgc

(19) United States
(12) Patent Application Publication
(10) Pub. No.: US 2017/0073736 A1
(43) Pub. Date: Mar. 16, 2017

(54) NUCLEIC ACID AMPLIFICIATION TECHNIQUES AND METHODS FOR DETECTING BACTERIAL INFECTION

(71) Applicant: BOARD OF REGENTS, THE UNIVERSITY OF TEXAS SYSTEM, Austin, TX (US)

(72) Inventors: Douglas Bryan LITWIN, Houston, TX (US); Heidi B. KAPLAN, Houston, TX (US); Catherine G. AMBROSE, Houston, TX (US); Anahita DUA, Brookfield, WI (US); John B. HOLCOMB, Houston, TX (US); Charles E. WADE, Houston, TX (US); Fernando ANDRADE, Sugarland, TX (US); John Darrell LIU, Sugarland, TX (US)

(21) Appl. No.: 15/224,116

(22) Filed: Jul. 29, 2016

Related U.S. Application Data
(60) Provisional application No. 62/198,207, filed on Jul. 29, 2015.

(51) Int. Cl. C12Q 1/68

(52) U.S. Cl. CPC C12Q 1/689; C12Q 2600/158

(57) ABSTRACT

Sequence specific DNA amplification and analysis techniques are provided. In some aspects, methods of the embodiments comprise amplifying sequence from two regions of a target sequence in the presence of a blocking oligonucleotide (e.g., such as a phosphorothioate-containing oligonucleotide) that hybridizes to the target sequence between the two regions. In some specific embodiments, a method is provided for detecting bacteria (such as detecting gram-positive or gram-negative bacteria) in a biological sample using polymerase chain reaction (PCR).

This application claims the benefit of U.S. Provisional Patent Application No. 62/198,207, filed Jul. 29, 2015, the entirety of which is incorporated herein by reference.

### INCORPORATION OF SEQUENCE LISTING

The sequence listing that is contained in the file named “UTSHP0312US_ST25.txt”, which is 8 KB (as measured in Microsoft Windows®) and was created on Jul. 27, 2016, is filed herewith by electronic submission and is incorporated by reference herein.

### BACKGROUND OF THE INVENTION

1. Field of the Invention

The present invention relates generally to the field of molecular biology and microbiology. More particularly, it concerns improved methods for nucleic acid amplification and for detection of bacterial infection.

2. Description of Related Art

Sepsis is a severe and lethal health problem that is a growing concern with respect to patient outcomes and financial considerations. The current clinical ‘gold standard’ technique of blood culture and Gram-stain analysis used to diagnose sepsis has many shortcomings. Many molecular methods for the identification of pathogens associated with sepsis are being developed. These methods that generally focus on rapid and accurate pathogen identification include techniques such as NMR, microscopy, PCR, microarrays and mass spectroscopy. To date, no rapid and reliable USFDA approved test for detecting and characterizing bacteria has been developed that could be applied in the USA to properly diagnose sepsis in a clinical setting.

### SUMMARY OF THE INVENTION

In a first embodiment there is provided a method for detecting bacteria in a biological sample from a subject. In some aspects, a method further comprises determining whether bacteria present in a sample are gram-negative or gram-positive bacteria. Thus in some aspects, a method of the embodiments comprises (a) amplifying at least two regions of a sequence encoding bacterial ribosomal RNA (rRNA) to generate at least two bacterial amplicons, wherein at least one of the two regions comprise sequences that are common to all bacteria (sequences that are shared between gram-positive and gram-negative bacteria) and sequences that are different between gram-positive and gram-negative bacteria; (b) hybridizing each of the at least two amplicons with at least one probe to produce a detectable signature, wherein the probe that hybridizes to one of the amplicons is specific for gram-positive or gram-negative bacterial sequences and the probe that hybridizes to the other of the amplicons is specific for bacterial rRNA sequences (but is non-specific for gram-positive or gram-negative bacterial sequences). In some aspects, a method involves hybridizing the amplicons to at least three hybridization probes, wherein two of the probes are specific for gram-positive or gram-negative bacterial sequences and the third probe is specific for bacterial rRNA sequences (but is non-specific for gram-positive or gram-negative bacterial sequences). In yet a further aspect, a method involves hybridizing the amplicons to at least four hybridization probes, wherein three of the probes are specific for gram-positive or gram-negative bacterial sequences and the fourth probe is specific for bacterial rRNA sequences (but is non-specific for gram-positive or gram-negative bacterial sequences). Thus, in some aspects, methods of the embodiments involve the use of four hybridization probes. For example, the probes can comprise a first probe that hybridizes to a first amplicon and is specific for bacterial rRNA sequences (but is non-specific for gram-positive or gram-negative bacterial sequences); a second probe that hybridizes to the first amplicon and is specific to gram-positive bacteria; a third probe that hybridizes to a second amplicon and is specific to gram-positive bacteria; and a fourth probe that hybridizes to the second amplicon and is specific to gram-negative bacteria. Thus, in some aspects, a method comprises (b) hybridizing each of the at least two amplicons with at least two probes to produce a detectable signature, wherein one of said probes that hybridizes to each of the amplicons is specific for gram-positive or gram-negative bacterial sequences and one of said probes that hybridizes to one the amplicons is specific for bacterial rRNA sequences (but is non-specific for gram-positive or gram-negative bacterial sequences).

In a related embodiment, an assay method is provided comprising (a) amplifying at least two regions of a sequence encoding bacterial ribosomal RNA (rRNA) from a biological sample from a subject to generate at least two bacterial amplicons, wherein at least one of the two regions comprise sequences that are shared between gram-positive and gram-negative bacteria and sequences that are different between gram-positive and gram-negative bacteria; and (b) hybridizing each of the at least two amplicons with at least two probes to produce a detectable signature, wherein at least one of said probes that hybridizes to each of the amplicons is specific for gram-positive or gram-negative bacterial sequences and one of said probes that hybridizes to one of the amplicons is non-specific for gram-positive or gram-negative bacterial sequences.

Thus, in further aspects, a method of the embodiments comprises determining whether bacteria in a sample are gram-positive or gram-negative bacteria. In still further aspects, a method comprises determining the type (e.g., the genus and/or species) of bacteria in a sample. In some aspects, a method can be used to detect the presence of Staphylococcus aureus, Enterococcus faecalis, Pseudomonas aeruginosa and/or Escherichia coli bacteria in a sample. In still further aspects, a method is defined as a method for detecting pathogenic bacteria. In some aspect, methods of the embodiments can be used for detecting 10,000, 5,000, 1,000, 500, 100 or less bacterial cells per ml of sample (e.g., blood sample) volume. In certain aspects, a method of the embodiments (e.g., all of the steps of the method) is performed over a time period no longer than 24, 12, 8, 6 or 5 hours.

Samples for use according to the embodiments can include any sample having or suspected of having bacteria present. For example, the sample can be a solid tissue sample, a fecal, urine, saliva, lymph or blood sample. In certain aspects the sample is a blood sample, such as a sample from a subject who has or is suspected of having sepsis.

Certain aspects of the embodiments involve identifying a sample (or a subject) as having bacteria present. In some aspects, a method involves identifying the presence of gram-positive or gram-negative bacteria in the sample (or a subject). In yet further aspects, a method comprises diagnosing a subject with bacterial sepsis (e.g., from gram-positive or gram-negative bacteria). In further aspects, a method comprises reporting the presence of bacteria or of gram-positive or gram-negative bacteria in the sample. For example, the reporting can be reporting to a doctor, a hospital, an insurance company or to the subject (from whom the sample originated). In further aspects, a method of the embodiments comprises preparing a written, oral or electronic report.

As detailed above, in certain aspects, methods of the embodiments comprise amplifying at least two regions of a sequence encoding bacterial rRNA to generate at least two bacterial amplicons. In some aspects, the two regions encode bacterial 23S or 16S rRNA or the regions of DNA between these coding regions. In further aspects, the amplification can comprise a reverse transcription step (e.g., to detect bacterial RNA). In still further aspects, the amplification is by quantitative PCR, such as quantitative real time PCR. In some aspects, at least one of the two regions comprise sequences that are common to all bacteria (sequences that are shared between gram-positive and gram-negative bacteria) and sequences that are different between gram-positive and gram-negative bacteria. In further aspects, each of the two regions comprise sequences that are common to all bacteria (sequences that are shared between gram-positive and gram-negative bacteria) and sequences that different between gram-positive and gram-negative bacteria. In certain specific aspects, one of the at least two regions of a sequence is amplified by primer pairs 16S557(19)F/16S786(23)R or primer pairs 16S945(20)F/16S1222(20)R. For example, in some cases, the at least two regions of a sequence are amplified by primer pairs 16S557(19)F/16S786(23)R and 16S945(20)F/16S1222(20)R. Thus, in certain aspects, a method of the embodiments involves amplifying a sequence by the primer pairs 16S557(19)F/16S786(23)R and/or 16S945(20)F/16S1222(20)R.

In some cases a sample may be directly obtained from a subject or may be obtained from a third party (e.g., a doctor or hospital). Samples may be processed, for example to lyse cells and/or purify or partially purify nucleic acids from the sample. In some aspects, a method may further involve pretreating reagents for use in the amplifying step with ethidium monoazide (EMA) or propidium monoazide (PMA).

Aspects of the embodiments involve the use of hybridization probes. In some aspects, at least one, two, three or more of the probes is/are fluorescently labeled. In further aspects, at least one, two, three or more of the probes is/are a minor-groove binding probe (e.g., comprising a 3′ dihydrocyclopyrroloindole tripeptide (DPI3)). In certain very specific aspects, one or more of the probes is selected from the groups consisting of P16S683U, P16S706P, P16S1194P, P16S1194N, P16S1195P, and P16S1195N.

In certain preferred aspects, a method comprises addition (e.g., prior to or during amplification) of at least one blocking oligonucleotide that includes at least one phosphorothioate position. In some aspects, a blocking oligonucleotide hybridizes to the DNA sequence encoding bacterial rRNA between two regions of the regions being amplified. In these aspects, the phosphorothioate modification(s) block the 5′ to 3′ exonuclease activity of the DNA polymerase and terminate extension (e.g., to prevent synthesis of an amplicon including sequences from both of the two regions).

In still a further embodiment, there is provided a method of treating a subject having sepsis comprising administering an antibiotic to a subject who was determined to have a bacterial infection by a method in accordance with the embodiments provided herein. For example, in some aspects, a method comprises administering an antibiotic effective against gram-positive bacteria, wherein the subject was determined to have an infection with gram-positive bacteria by a method of the embodiments. Conversely, in some aspects, a method comprises administering an antibiotic effective against gram-negative bacteria, wherein the subject was determined to have an infection with gram-negative bacteria by a method in accordance with the embodiments.

In still a further embodiment, there is provided a method for sequence-specific amplification of at least two regions of a target nucleic acid sequence comprising performing PCR amplification of at least two regions of a target nucleic acid sequence using primer pairs specific to the at least two regions and at least one blocking oligonucleotide that includes at least one phosphorothioate position, wherein said blocking oligonucleotide hybridizes to the target nucleic acid sequence between two of said at least two regions of sequence. In some aspects, a method further comprising detecting a sequence amplified from said at least two regions using at least two sequence-specific hybridization probes (e.g., fluorescently labeled probes).

In certain aspects, a blocking oligonucleotide of the embodiments includes at least 2, 3, 4, 5, 6, 7 or 8 phosphorothioate positions. In some aspects, a blocking oligonucleotide includes at least one phosphorothioate position at the 5′ end of the oligonucleotide. For example, a blocking oligonucleotide can comprise at least 2, 3, 4, 5, 6, 7 or 8 consecutive phosphorothioate positions beginning at the 5′ end of the oligonucleotide. In still further aspects, a blocking oligonucleotide comprises 1, 2, 3, 4, 5, 6, 7, 8, 9 or 10 mismatches (e.g., positions at or near the 3′ end of the oligonucleotide) relative to the target nucleic acid sequence. In further aspects, a blocking oligonucleotide comprises a region of high GC content that is complementary to the target sequence (e.g., a region with a GC content of at least 50%, 60%, 70°/o or 80%). Thus, in certain specific aspects, a blocking oligonucleotide comprises, from 5′ to 3′, (i) 3 to 8 phosphorothioate positions: (ii) a GC clamp comprising sequence complementary to the target sequence and having a GC content of greater than 60%; and (iii) a 3′ sequence including 3 to 8 mismatches relative to the target sequence. In some aspects, the 3′ sequence comprises 1, 2, 3, 4, 5 or more A nucleotides.

In still a further embodiment there is provided a kit comprising at least two primer pairs suitable for amplification of two regions of sequence encoding bacterial ribosomal RNA (rRNA), wherein at least one of the two regions comprise sequences that are shared between gram-positive and gram-negative bacteria and sequences that are different between gram-positive and gram-negative bacteria; and (ii) at least three hybridization probes, wherein two of the probes are specific for gram-positive or gram-negative bacterial sequences and one of the probes is non-specific for gram-positive or gram-negative bacterial sequences. In further aspects, a kit comprises at least four hybridization probes, wherein three of the probes are specific for gram-positive or gram-negative bacterial sequences and one of the probes is non-specific for gram-positive or gram-negative bacterial sequences. In yet further aspects, two of the four probes hybridize to a sequence corresponding to one of the two regions of sequence encoding bacterial rRNA and the other two of the four probes hybridize to a sequence corresponding to the other of the two regions of sequence encoding bacterial rRNA. In still further aspects, a kit comprises at least one blocking oligonucleotide that includes at least one phosphorothioate position, wherein said oligonucleotide hybridizes to the sequence encoding bacterial rRNA between the two regions of the sequence encoding bacterial rRNA.

In still a further embodiment there is provided a kit comprising (i) at least two primer pairs suitable for amplification of two non-overlapping regions of a target sequence; and (ii) at least one blocking oligonucleotide that includes at least one phosphorothioate position, wherein said oligonucleotide hybridizes to the sequence between the two non-overlapping regions of the target sequence. In some aspects, a blocking oligonucleotide includes at least one phosphorothioate position (e.g., at least 2, 3, 4, 5, 6, 7 or 8 consecutive phosphorothioate positions) at the 5′ end of the oligonucleotide. In further aspects, the blocking oligonucleotide comprises 1, 2, 3, 4, 5, 6, 7, 8, 9 or 10 mismatches (e.g., located at or near the 3′ end of the blocking oligonucleotide) relative to the target nucleic acid sequence. In still further aspects, a kit comprises at least one hybridization probe that hybridizes to a sequence corresponding to one of the two non-overlapping regions.

In some embodiments are methods that can be used to differentiate patients, with the signs or symptoms of sepsis, which will not benefit from treatment with antibiotics from those patients that will. In other embodiments are methods of identifying a subject with one or more signs or symptoms of sepsis that is in need of treatment with antibiotics. In additional embodiments are methods of excluding the use of antibiotic therapy for a subject with one or more signs or symptoms of sepsis.

In some embodiments, the present method utilizes hybridization of nucleic acid probes to pathogen specific bacterial DNA during PCR amplification, as an indicator of the presence of bacteria in biological samples obtained from patients who appear to be septic. The absence of an amplification product indicates that the patient is not septic due to infection with bacteria and that such patients will not benefit from antibiotic therapies directed to such bacteria. Thus, as a result of this method only those patients with the signs or symptoms of sepsis who will benefit from antibiotic therapy will receive it. This avoids unnecessary expense, exposure to adverse effects of antibiotic therapy and should also minimize the induction of antibiotic resistant organisms.

In some aspects, a hybridization probe according to the embodiments includes a reporter. Numerous reporter molecules that may be used to label nucleic acids are known. Direct reporter molecules include fluorophores, chromophores, and radiophores. Non-limiting examples of fluorophores include, a red fluorescent squarine dye such as 2,4-Bis[1,3,3-trimethyl-2-indolinylidenemethyl]cyclobutenediylium-1,3-dio-xolate, an infrared dye such as 2,4 Bis[3,3-dimethyl-2-(1H-benz[e]indolinylidenemethyl)]cyclobutenediylium-1,-3-dioxolate, or an orange fluorescent squarine dye such as 2,4-Bis[3,5-dimethyl-2-pyrrolyl]cyclobutenediylium-1,3-diololate. Additional non-limiting examples of fluorophores include quantum dots, Alexa Fluor™ dyes, AMCA, BODIPY™ 630/650, BODIPY™ 650/665, BODIPY™-FL, BODIPY™ R6G, BODIPY™-TMR, BODIPY™-TRX, Cascade Bluen™, CyDye™, including but not limited to Cy2™, Cy3™, and Cy5™, a DNA intercalating dye, 6-FAM™, Fluorescein, HEX™, 6-JOE, NED®, VIC™, Oregon Green™ 488, Oregon Green™ 500, Oregon Green™ 514, Pacific Blue™, REG, phycobilliproteins including, but not limited to, phycoerythrin and allophycocyanin, Rhodamine Green™, Rhodamine Red™, ROX™, TAMRA™, TET™, Tetramethylrhodamine, or Texas Red™. In some aspects, a signal amplification reagent, such as tyramide (PerkinElmer), may be used to enhance the fluorescence signal. Indirect reporter molecules include biotin, which must be bound to another molecule such as streptavidin-phycoerythrin for detection. Pairs of labels, such as fluorescence resonance energy transfer pairs or dye-quencher pairs, may also be employed according to the embodiments.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1. Schematic of the Early Molecular Detection assay. The EMD assay targets the bacterial 16S rRNA gene. The upstream amplicon is comprised of primers and probes; 16S557(19)F, 16S786(23)R, P16S683U, and P16S706P. The downstream target is composed of primers and probes; 16S945(20)F, 16S1222(20)R, P16S1194P, and P16S1194N.

FIG. 2. Nucleotide alignments of the 16S rRNA genes from the STICU pathogen list. Species are grouped as gram-positive and gram-negative. Areas identified as ideal targets for gram-negative identification are shown in red. The area identified as ideal targets for gram-positive identification is shown in blue. Areas identified as ideal targets for universal bacterial identification are shown in green.

FIGS. 3A-B. Alignments with the reverse complementation of the universal probe P16S683U. (A) Reverse complementation of P16S683U (SEQ ID NO: 1) aligned with STICU gram-negative species. (B) Reverse complementation of P16S683U (SEQ ID NO: 1) aligned with STICU gram-positive species. Nucleotide mismatches with the probe sequence are bolded.

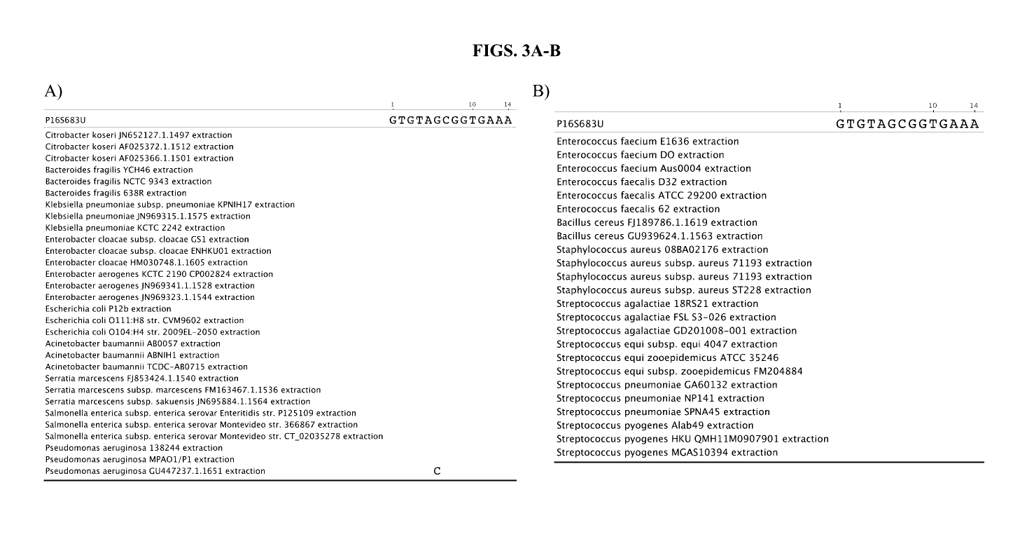

FIGS. 4A-B. Alignments with the reverse complementation of the gram-positive probe P16S706P. (A) Reverse complementation of P16S706P (SEQ ID NO: 2 or 3) aligned against STICU gram-negative species. (B) Reverse complementation of P16S706P (SEQ ID NO: 2 or 3) aligned against STICU gram-positive species. Nucleotide mismatches with the probe sequence are bolded.

FIGS. 5A-B. Alignments with the reverse complementation of the gram-negative probe P16S1194N. (A) Reverse complementation of P16S1194N (SEQ ID NO: 4) aligned against STICU gram-negative species. (B) Reverse complementation of P16S1194N (SEQ ID NO: 4) aligned against STICU gram-positive species. Nucleotide mismatches with the probe sequence are bolded.

FIGS. 6A-B. Alignments with the reverse complementation of the gram-positive probe P16S1194P. (A) Reverse complementation of P16S1194P (SEQ ID NO: 5) aligned against gram-negative species. (B) Reverse complementation of P16S1194P (SEQ ID NO: 5) aligned against STICU gram-positive species. Nucleotide mismatches with the probe sequence are bolded.

FIG. 7. An electrophoresis gel analysis of different primer-pair efficiencies. Lane 1) Primers 16S560(18)F and 16S786(18)R, Lane 2) Primers 16S560(18)F and 16S786(23)R, Lane 3) Primers 16S557(19)F and 16S786(18)R Lane 4) Primers 16S557(19)F and 16S786(23)R.

FIGS. 8A-B. Standard curve analysis of upstream primers 16S557(19)F and 16S786(23)R. (A) Primers 16S557(19)F and 16S786(23)R amplifying gram-positive species S. aureus and E. faecalis. (B) Primers 16S557(19)F and 16S786(23)R amplifying gram-negative species E. coli and P. aeruginosa.

FIGS. 9A-B. Standard curve analysis of downstream primers 16S945(20)F and 16S1222(20)R. (A) Primers 16S945(20)F and 16S1222(20)R amplifying gram-positive species S. aureus and E. faecalis. (B) Primers 16S945(20)F and 16S1222(20)R amplifying gram-negative species E. coli and P. aeruginosa.

FIGS. 10A-B. Fluorescent detection and standard curve plots for the upstream amplicon. A) Probe 16S683U analyzing E. coli, P. aeruginosa, S. aureus and E. faecalis DNA. B) Probe P16S706P analyzing E. coli, P. aeruginosa, S. aureus and E. faecalis DNA.

FIGS. 11A-B. Fluorescent detection and standard curve plots for the downstream amplicon. A) Probe P16S1194P analyzing E. coli, P. aeruginosa, S. aureus and E. faecalis DNA. B) Probe P16S1194N analyzing E. coli, P. aeruginosa, S. aureus and E. faecalis DNA.

FIGS. 12A-B. Analysis of competitive binding for downstream probes. A) Probe P16S1194P analyzing E. faecalis DNA in singleplex and duplex reactions with probe P16S1194N. B) Probe P16S1194N analyzing E. coli in singleplex and duplex reactions with probe P16S1194P.

FIG. 13. Effects of EMA treatment on qPCR reaction. Three log dilutions of S. aureus DNA with and without EMA pretreatment of qPCR mastermix. Samples with EMA pretreatment are shown in blue. Samples without EMA pretreatment are shown in green. The reagents alone without EMA treatment is shown in orange. The reagents alone with EMA treatment is shown in red. These qPCR reactions contained the upstream primers 16S557(19)F and 16S786(23)R and the P16S706P probe.

FIGS. 14A-B. The use of phosphorothioate blocking oligonucleotides to prevent the generation of large PCR amplicons that arise from the amplification of products from outlying primers that occur in multiplex PCR reactions with targets in close proximity. (A) Two PCR targets in close proximity facilitate the generation of a large PCR amplicon by amplification initiating from the outlying primers (UpFp and DnRp). (B) The phosphorothioate blocking oligonucleotide anneals to the template downstream of the upstream forward primer and terminates extension.

FIGS. 15A-B. 5′ to 3′ Phosphodiester linkage and 5′ to 3′ phosphorothioate linkage. (A) A phosphodiester linkage contains two non-bridging oxygens (one is shown in red). (B) A phosphorothioate linkage contains a non-bridging oxygen and a non-bridging sulfur shown in red.

FIGS. 16A-D. A model of the phosphorothioate barrier to PCR amplification. (A) The phosphorothioate blocking oligonucleotide anneals to the template downstream of the upstream forward primer. The DNA polymerase extends from the upstream forward primer toward the blocking oligo. (B) and (C) The polymerase encounters the blocking oligo and is unable to hydrolyze the phosphorothioate-modified nucleotides. The extension is terminated and the polymerase detaches from the DNA. (D) If the blocking oligo lacking the phosphorothioate modifications anneals to the template, the 5′ to 3′ exonuclease activity of polymerase removes the nucleotides and extension is completed.

FIG. 17. The use of phosphorothioate blocking oligonucleotides to prevent the generation of large PCR amplicons that arise from the amplification of products from outlying primers that occur in multiplex PCR reactions with targets in close proximity. An agarose gel shows the presence of the large flanking amplicon resulting from amplification from the outlying primers in the reaction in which the blocking oligonucleotide lacked the phosphorothioate linkage modifications and elimination of the large amplicon in the reaction with the phosphorothioate blocking oligo.

FIGS. 18A-B. Fluorescent detection and standard curve plots for the downstream amplicons using the probes P16S1195P(VIC) and P16S1195N(6FAM). (A) Probe P16S1195P analyzing E. coli and S. aureus DNA at 1×10⁵ to 1×10² cell equivalents. All of the E. coli DNA amplification is below the threshold value. (B) Probe P16S1195N analyzing E. coli, and S. aureus DNA at 1×10⁵ to 1×10² cell equivalents. All of the S. aureus DNA amplification is below the threshold value.

FIG. 19. Schematic of plating of DNA standards as described in Example 6.

### DESCRIPTION OF ILLUSTRATIVE EMBODIMENTS

### I. The Present Invention

Sepsis is a severe and lethal health problem that is a growing concern with respect to patient outcomes and financial considerations. The current clinical ‘gold standard’ technique of blood culture and Gram-stain analysis used to diagnose sepsis has many shortcomings. Many molecular methods for the identification of pathogens associated with sepsis are being developed. These methods that generally focus on rapid and accurate pathogen identification include techniques such as NMR, microscopy, PCR, microarrays and mass spectroscopy.

Provided is a quantitative real-time PCR assay to detect and classify based on the Gram status of 33 of the bacteria commonly recovered from sepsis patients. This EMD assay was designed to include internal checks and balances to provide a more accurate detection technique. In some embodiments, the assay utilizes a dual amplicon approach with two probes per amplicon to detect all bacterial DNA by hybridization to a universally conserved region and to differentiate gram-positive and gram-negative bacterial DNA by hybridization to regions specific to these two bacterial groups.

The EMD assay was directly developed using bacterial DNA. Subsequently, the entire protocol from DNA extraction through analysis was tested in samples in which each one of the four representative laboratory strains were independently added in a range of five orders of magnitude concentrations (10⁵ to 10¹) to previously uninfected human blood samples and phosphate buffered saline (PBS). Finally, 18 clinical samples from patients suspected to have sepsis were analyzed by the EMD assay and the Memorial Hermann Hospital Clinical Microbiology Laboratory and the results were compared.

The assay is capable of accurately measuring the concentration of bacterial DNA of S. aureus, E. faecalis, P. aeruginosa and E. coli extracted from blood and PBS with amplification efficiencies from 70-90%. The universal probe P16S683U detected DNA of all of the four laboratory strains that were tested and was determined by in silico analysis to identify many other genera and species. Three gram-positive probes, P16S706P, P16S1194P and P16S1195P, detected both of the gram-positive species without detecting the two gram-negative species. In addition, in silico analysis determined that the probes could detect many other gram-positive genera and species, but not gram-negative species. Two gram-negative probes, P16S1194N and P16S1195N, detected both of the gram-negative species without detecting the two gram-positive species and in silico analysis determined that they detected many other gram-negative genera and species, but not gram-positive species.

The EMD assay analysis of the 18 clinical samples was compared to results obtained from the Memorial Hermann Hospital Clinical Microbiology Laboratory. There was 100% congruence between the two positive blood culture results and the EMD assay results. In both cases the pathogens identified were gram-negative bacteria. There was 37% congruence between the 16 culture negative samples and the EMD assay results, as six of these samples were also detected as amplification negative by the EMD assay or the results were detected below the 100-cell/ml threshold limit of detection. There was 62% non-congruence between the culture negative samples and the EMD assay results, as ten of the 16 culture negative samples were amplification positive by the EMD assay.

One of the advantages of the EMD method over other molecular detection methods is that the EMD method can ‘rule out’ infection as the cause of the patient's symptoms. This is possible due to the inclusion in the assay of the universal probe that should bind to any bacteria DNA in the sample. All other assays developed to date are of a ‘rule in’ type in which the method is used to determine if the sample contains a pathogen included in the list of organisms that can be detected by the method. As a result, if a bacterium out of the group is the causative organism it cannot be ruled out. As a result physicians prefer to adhere to the use of blood culture results. However, it is clear that blood culture is slow and not a definitive assay, as many bacteria do not grow well enough to reach the approximately 10⁵ cell/ml requirement and it is clear by the high number (62%) of samples that are negative by culture but resulted in a positive amplification.

The EMD assay had a faster time to detection (TTD), which was estimated as 4.5 hr from sample arrival in the laboratory through analysis. The positive blood culture and Gram-stain analysis required an average of 20 hr until the information was transmitted to the physician. However, when the blood culture analysis was negative the TTD was 5-7 days. Although blood culturing is the current ‘gold standard’ in pathogen detection in blood samples, it appears that it should not be used as a benchmark for molecular methods of diagnosis.

To differentiate live from dead bacteria in the clinical blood samples, all of the blood samples were subject to centrifugation after collection and the pellet and supernatant were stored separately at −20° C. until analysis. The plasma DNA for samples 8 to 18 were also analyzed.

In some embodiments there is provided an Early Molecular Detection assay to identify the Gram status of sepsis-associated bacterial pathogens. The assay contains internal checks and balances, detects all bacteria by hybridization to a universally conserved DNA region, and differentiates Gram status by hybridization to specific DNA regions.

An important concern associated with using the 16S rRNA gene as an assay target is that the assay is so sensitive that it can detect any bacterial DNA that is present and essentially contaminating the PCR and DNA extraction solutions. To address this EMA- or PMA-treated H₂O is used to make the dilutions of the probes from the 100 uM and the 10 uM working stocks. The probes were then added to the master mix after the EMA treatment of the qPCR master mix. This eliminated the exposure of the probes to EMA crosslinking and prevented the addition of detectable contaminant DNA.

One surprising advantage to the extraction of small quantities of bacterial DNA from blood samples is that the concurrent extraction of human DNA serves as carrier DNA that increases the yield of the bacterial DNA. Much greater yields of DNA from blood compared to PBS in the DNA extracted and analyzed were noted when each of the four laboratory strains were tested in blood samples and PBS. The results from the DNA extracted from blood samples were typically within one log of the input cell concentration used, whereas the DNA extracted from PBS were consistently 2-logs lower than the input cell concentration. The carrier DNA can also serve to dilute the bacterial DNA during the DNA extraction procedure and therefore any small percentage loss in yield would have significantly less impact on the overall amount of bacterial DNA that is lost. As a result, DNA extraction from PBS was modified by adding 200 ng of yeast genomic DNA to act as carrier DNA. This improved the detection of our control organisms significantly.

It was noted during EMD analysis that the S. aureus DNA extracted from the blood was always one order of magnitude less than the input cells, whereas all of the other three organisms were much more closely correlated to the amount of input cells. It was determined that the EMA-treated PBS used in the washing and dilution steps was killing the S. aureus cells (see, e.g., Table 4). After switching to PMA for decontamination of the solutions to which the live S. aureus cells were exposed, more consistent results were observed for S. aureus. It is important to note that although this was important in these control experiments in which live cells were added to blood and PBS, this is not relevant to clinical samples as the DNA is already extracted from the cells as a first step.

The EMD assay was focused on the Gram status identification of the 33 most frequently recovered bacterial species in presumed septic patients in the STICU of Memorial Herman Hospital in Houston, Tex. It is certain that different pathogens would be more common in different types of infections. One advantage of the EMD assay is that it is easily amendable to species not covered in its current configuration due to the low variability of the probe target sites. It is expected that by adding a mixture of degenerate probes containing single or double nucleotide substitutions for the gram-positive and gram-negative probes, with all degenerate variations using the same fluorophore, the assay's species coverage could be greatly increased. However, this addition to the assay requires experimental validation.

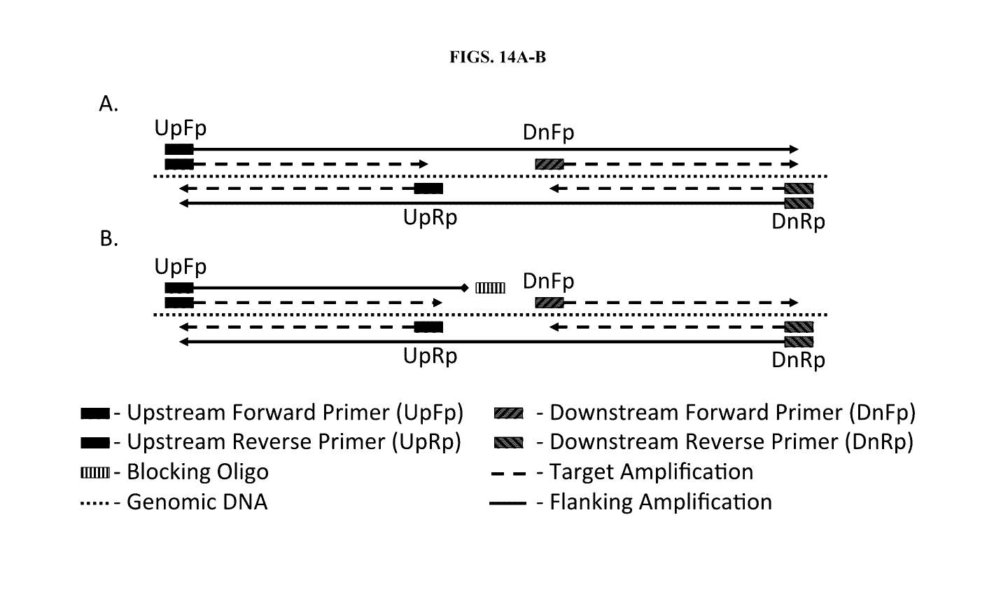

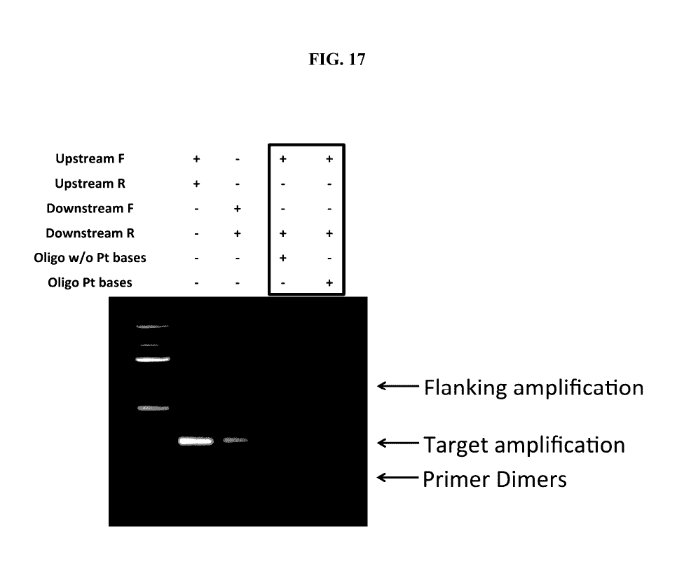

The EMD assay is currently configured as two duplex reactions that are compared as ‘upstream’ and ‘downstream’ reactions in separate wells of a 96 well assay plate. However in some embodiments the EMD assay is configured as a quadruplex reaction in which all four primers and all four probes are present in one well. This configuration creates a situation in which the extension from the forward primer from the upstream amplicon and the reverse primer from the downstream amplicon will generate a PCR product that is termed the ‘flanking amplification’ product (FIG. 14). These flanking products are of particular concern because they are anticipated to use an abundance of reaction components causing the amplification of the target amplicons to be less efficient. To address this concern, a ‘blocking’ oligonucleotide containing phosphorothioate modifications is used that will anneal between the upstream reverse primer and the downstream forward primer. This blocking oligonucleotide will function to stop the extension of polymerase by forming a stable duplex with the target site and preventing hydrolysis of the blocking oligo by the phosphorothioate modifications. This technique has been tested in standard PCR and shows great promise for further use (FIG. 17).

Described is a molecular diagnostic technology for the detection of sepsis-associated infections that decreases the time to pathogen detection. This molecular technology is an improvement upon the current culture-based methods in that it is more rapid, sensitive and specific.

The current new techniques to diagnose infections include PCR and variations of PCR analysis, mass spectroscopy, automated microscopy, NMR probe detection. The described EMD assay is a rapid, specific and sensitive molecular assay for the culture-independent detection of bacterial pathogens in blood samples from suspected sepsis patients.

The bacterial 16S rRNA gene is used as a target for the identification of bacterial genera and species and the EMD assay utilizes this feature as a method to detect the bacteria and to differentiate their Gram status. However, it is not necessary to limit the use of this assay to detection alone. The two amplicons (229 bp and 270 bp) can also be subjected to DNA sequence analysis, which should be useful in identification of the genus and possibly species of the pathogen particularly if it is a single or predominant organism present in the blood sample. The use of next generation DNA sequences techniques should allow the identification of bacteria in multi-species infections.

Furthermore, there are many options for downstream species identification with species-specific probes including those that can identify the DNA of fungal pathogens for use in combination with the EMD assay.

### II. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Determination of Relevant Organisms

A list of the 33 bacterial species most frequently found in positive blood cultures in the surgical trauma intensive care unit (STICU) from 2009-2012 at Memorial-Herman Hospital was obtained from the STICU pharmacy (Table 1). An additional group of 90 bacterial species assembled for an osteomyelitis study previously performed in our laboratory, was used for in silico analysis to test the extent of our probe coverage for bacterial pathogens in general.

TABLE 1
List of the 33 most frequent bacterial species recovered in the
Memorial-Herman Hospital STICU from 2009-2012.
Species are grouped by Gram status and listed by the
frequency of recovery for the 134 samples analyzed.

| gram-negative | | | Gram-Positive | | |
|---|---|---|---|---|---|
| Rank | Species | Frequency | Rank | Species | Frequency |
| 1. | E. coli | 11 | 1. | Staphylococcus | 28 |
| | | | | not aureus | |
| 2. | Bacteroides | 10 | 2. | MSSA | 10 |
| | fragilis | | | | |
| 3. | Enterobacter | 7 | 3. | Enterococcus | 7 |
| | cloacae | | | | |
| 4. | Klebsiella | 7 | 4. | MRSA | 6 |
| | pneumoniae | | | | |
| 5. | Pseudomonas | 7 | 5. | Alpha strep | 5 |
| | aeruginosa | | | | |
| 6. | Serrratia | 6 | 6. | Bacillus sp. | 2 |
| | marcesens | | | | |
| 7. | Acinetobacter | 5 | 7. | Gamma strep not | 2 |
| | baumannii | | | ent | |
| 8. | Acinetobacter | 1 | 8. | Group A beta | 2 |
| | lwoffi | | | strep | |
| 9. | Citrobacter | 1 | 9. | Group C strep | 2 |
| | koserii | | | | |
| 10. | Enterobacter | 1 | 10. | Bacillus cereus | 1 |
| | aerogenes | | | | |
| 11. | Eikenella | 1 | 11. | Cornybacterium | 1 |
| | corrodens | | | | |
| 12. | Enterobacter | 1 | 12. | Group B strep | 1 |
| | asburiae | | | | |
| 13. | Prevotella | 1 | 13. | Group F strep | 1 |
| 14. | Proteus | 1 | 14. | Group G strep | 1 |
| | mirabilis | | | | |
| 15. | Salmonella | 1 | 15. | Pepto- | 1 |
| | | | | streptococccus | |
| | | | 16. | Streptococcus | 1 |
| | | | | pneumoniae | |
| | | | 17. | Streptococcus | 1 |
| | | | | species | |
| | | | 18. | Vancomycin- | 1 |
| | | | | resistant | |
| | | | | Enterococcus | |

### Example 2

### Early Molecular Detection (EMD) Assay

EMA and PMA Treatment

Pretreatment of solutions containing the appropriate master mix and primers with either ethidium monoazide (EMA) or propidium monoazide (PMA) was required to eliminate contaminating bacterial DNA present in solutions that would otherwise be detected as a part of the sample. The PCR and qPCR master mixes used were either from the QuantiFast Probe PCR Kit or the QuantiFast SYBR Green PCR Kit (QIAGEN, Valencia, Calif.). Stock solutions of EMA and PMA at 239 μM and 20 mM, respectively were stored at −20° C. in the dark. In the presence of reduced light, EMA was added to the previously prepared master mix to a final concentration of 9 μM. The solution was briefly mixed by repeat pipetting, incubated at room temperature for 5 min, and exposed to a 500 watt light (Gulo Model A) from a distance of 10 cm for 5 min while on ice. Then the appropriate probes, (10 μM in EMA-treated H₂O) were added to a final concentration of 0.25 μM while the solution remained on ice. The same procedure was performed for the PMA treatment (20 μM final concentration) of PBS and the DNA extraction reagents.

Testing the qPCR Assay in Artificial Blood Samples

Blood was drawn from health volunteers into 4.5 ml citrate-containing Vacutainer tubes (Becton Dickinson). Aliquots (1 ml) of the blood were placed into 1.5 ml microcentrifuge tubes. Cultures (10 μl) of S. aureus, E. faecalis, P. aeruginosa and E. coli at concentrations two logs higher than the desired final concentration in the blood sample were added to the 1 ml aliquots to give final concentrations of 10⁵-10¹ CFU/ml. Those blood samples were then centrifuged at 13,000×g for 2 to 5 min, the plasma (upper phase) was removed and both fractions (the blood pellets and plasma) were stored at −20° C. until analysis. Similar control samples were also prepared in 20 μM PMA-treated PBS. Those samples were prepared by adding 10 μl of the bacterial culture, two logs higher than the desired concentration, and 2 μl of yeast genomic DNA (500 ng/μl) to a sterile 1.5 ml microcentrifuge tube containing 1 ml of PMA-treated PBS. The samples were stored at −20° C. until analysis. The total DNA was later extracted using the BiOstic® Bacteremia DNA Isolation kit (MO BIO Laboratories, Carlsbad, Calif.). Both the PBS and blood pellet extracted samples were then analyzed using the EMD assay.

Collection of Clinical Blood Samples

The clinical blood samples were drawn in sodium citrate-containing Vacutainers by nurses in the Memorial-Herman Hospital's STICU and Burn Unit based on a physician's diagnosis of a presumed septic patient. The blood-filled Vacutainers were maintained at room temperature until they were collected, given a sample number associated with the patent ID number, and transferred to the research laboratory by a laboratory manager generally within 12 hr of the blood draw. Aliquots (1 ml) of the blood from the Vacutainer were transferred into labeled sterile 2.0 ml microcentrifuge tubes, centrifuged at 13,000×g for 2 to 5 min, and the upper phase (plasma) was then removed without disturbing the blood pellet and transferred to a sterile 1.5 ml microcentrifuge tube. The blood pellets, plasma and remaining Vacutainer tubes were then stored at −20° C. until analysis.

Isolation of DNA from Blood Samples

DNA was isolated from blood and plasma samples using the BiOstic® Bacteremia DNA Isolation Kit according to the manufacturer's recommended protocol, except that reagents were pretreated, in some cases, with EMA or PMA. Briefly, the blood pellet was thawed and resuspended in solution CB1 (450 μl), transferred into a 2 ml microbead tube, briefly vortexed and heated to 80° C. for 15 min. The tubes were then horizontally secured onto a Mo-bio Vortex-Genie® attachment and vortexed at max speed for 10 min. The tubes were then centrifuged at 10,000×g for 1 min and the supernatant was transferred to a new 1.5 ml microcentrifuge tube. The CB2 solution (100 μl) was then added and the samples were vortexed briefly. The samples were incubated at room temperature for 5 min and then centrifuged at 10,000×g for 1 minute. The supernatant was transferred to a new 1.5 ml microcentrifuge tube. Solution CB3 (1 ml) was added and mixed by repeat pipetting. A portion of the solution (600 μl) was then added to a spin filter column and centrifuged at 10,000×g for 1 min. The run-through was discarded. This step was repeated until the entire sample was passed through the column. The column was then transferred to a new collection tube and washed twice with solution CB4 (600 μl) and centrifuged at 10,000×g for 1 min. The column was then centrifuged at 13,000×g for 3 min to dry the column. The DNA was then eluted using solution CB5 or H₂O (50 μl). The plasma samples were treated identically, except that twice the volume of reagents was used because of their larger volume.

qPCR of DNA from Blood Samples

The qPCR master mix contained QuantiFast Probe PCR+ROX Vial Kit (QIAGEN Valencia, Calif.), forward and reverse primers (0.5 uM) and H₂O. The complete master mix was treated with EMA (at a final concentration of 9 uM). Probes that were previously diluted to 10 uM in EMA-treated H₂O were added after the EMA treatment Aliquots (15 ul) of the completed master mix were then placed in a standard 96 well polypropylene qPCR plate (Denville Scientific Inc. Metuchen, N.J.). The samples (5 μl) were added and analyzed in duplicate and standards (5 μl) were used in concentrations representing 10⁵ to 10¹ cell/ml. The qPCR cycling conditions used were based on the manufacturer's recommendation: 1) initial denaturation step of 95° C. for 3 min, 2) denaturation step of 95° C. for 10 sec, 3) annealing step of 58° C. for 5 sec. and 4) extension step of 60° C. for 1 min. Steps 2-4 are then repeated 40 times.

The qPCR samples were then analyzed using the Applied Biosystems 7000 system software (Life Technologies, Grand Island, N.Y.). Quantitation of unknown samples was automatically calculated by standard curve comparison of Cₜ values.

Results

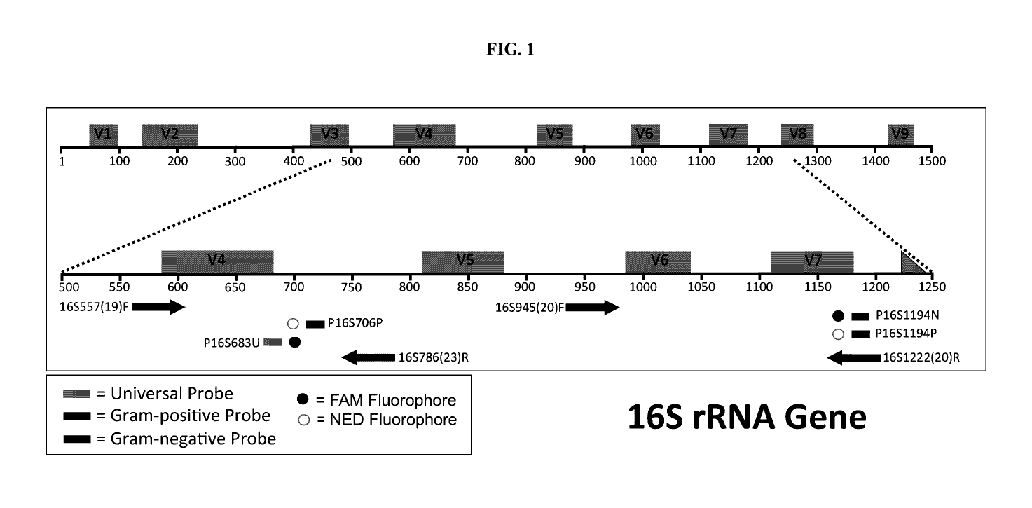

The EMD assay is a quantitative real-time PCR method for rapid, sensitive, and quantitative pathogen detection in blood samples from septic patients. Specifically, it can determine the Gram status of a pathogen is a blood sample within 4.5 hr. The use of minor groove binding probes makes it possible to have high binding fidelity with short target sequences. The EMD assay was designed to couple these highly specific probes with internal checks and balances. Specifically, the EMD assay is designed to amplify two regions of the bacterial 16S rRNA gene, which the inventors have termed the ‘upstream’ and ‘downstream’ amplicons. Two probes, a universal and a gram-positive probe, target the upstream amplicon and two probes, a gram-negative and gram-positive probe, target the downstream amplicon (FIG. 1). By using this dual amplicon approach, the assay detects bacterial pathogens and differentiates them by Gram status with great sensitivity.

Determination of Areas of Interest within the 16S rRNA Gene

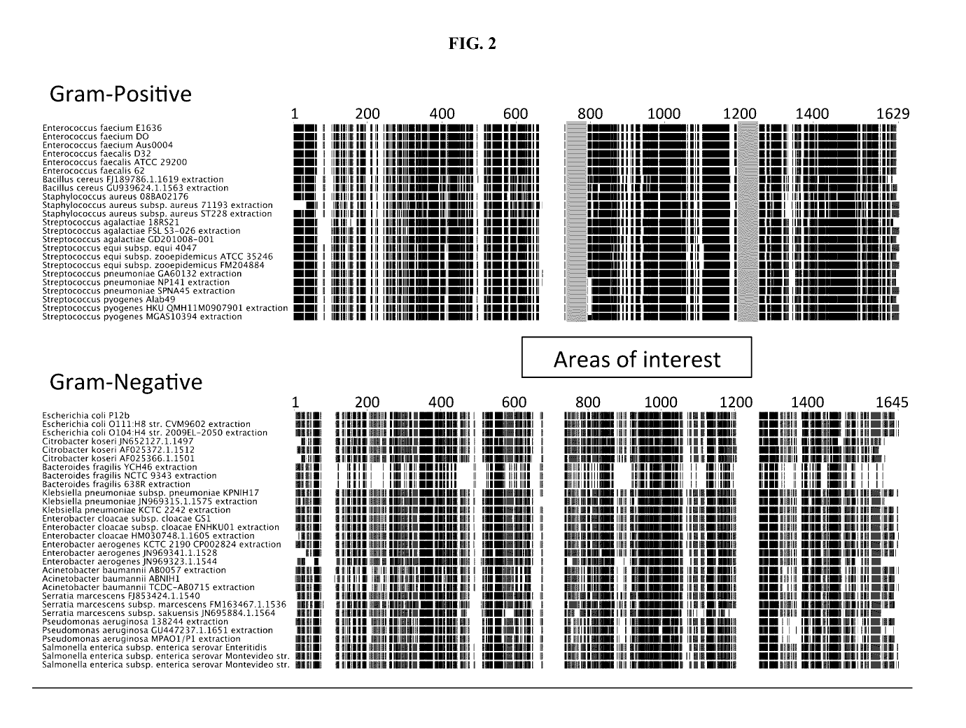

The bacterial 16S rRNA gene was chosen as the target of the EMD assay because it is present in all bacterial genomes in single or multiple copies and is composed of conserved and variable regions that can be used to differentiate species and genera. These genes are especially useful for probe-based qPCR assays because the conserved regions function to amplify the variable regions, which can serve as unique targets for differentiation by DNA probes. To identify the best regions in which to design our probes, three 16S rRNA genes chosen at random from each of the 33 most frequent STICU pathogens detected in septic patients in the Memorial Hermann Hospital STICU were aligned using the Geneious software suit (Biomatters Limited, San Francisco, Calif.)(CLUSTAL nucleotide alignment algorithm). The nucleotide alignments of the 33 relevant organisms were evaluated for use as probe target regions. Three types of conserved regions were characterized (FIG. 2): one universally conserved, two conserved among the gram-positive species, and one conserved among the gram-negative species.

Probe Design

To select specific areas highly conserved among the 18 gram-positive pathogens the sequences were aligned and compared to the same region in the 15 gram-negative pathogens. The same analysis was performed to select areas highly conserved among the gram-negative pathogens. Areas unique to either gram-positive or gram-negative pathogens or both were selected and evaluated for probe parameters using the AlleleID software suit (PREMIER Biosoft, Palo Alto, Calif.). Regions universally conserved (common to both the gram-positive and gram-negative pathogens) were also evaluated. Four gram-positive, two gram-negative and one universal probes listed below (Table 2) were identified that met our criteria. These criteria included: 1) the region spanned at least 13 nucleotides that maintained approximately 100% homology among all of the gram-positive, gram-negative, or all of the species in the alignments, 2) the sequence had more cytosine's than guanines and no 5′ guanine, and 3) at least one discriminatory mismatch was located near the 3′ terminus. The number of guanines is important as they can act as a fluorescent quencher, which suppresses the fluorescent signal available for detection in the qPCR reaction.

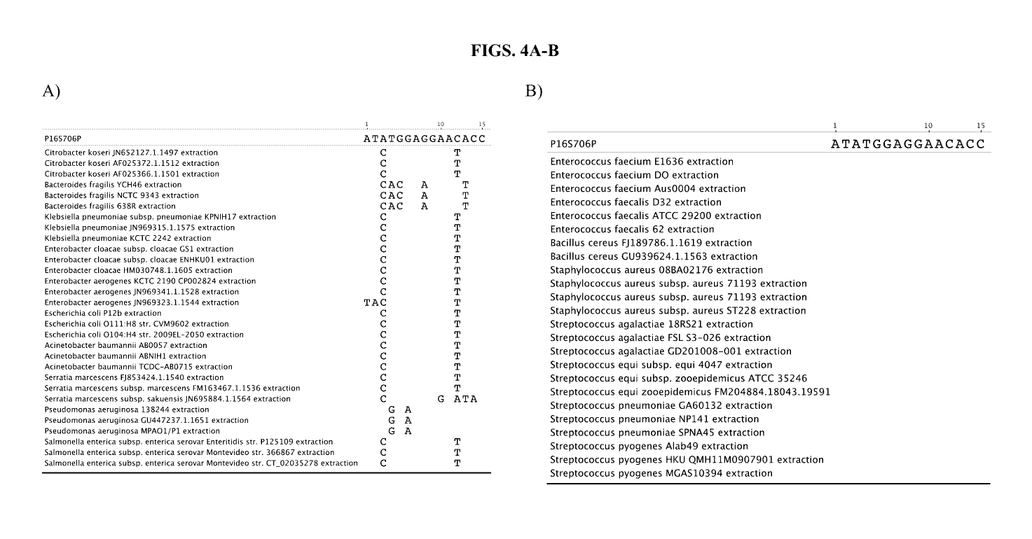

One region approximately 100% conserved among all the species and one region just downstream of that which is 100% conserved among the gram-positive species were identified (Table 2 and FIG. 2, 3, 4). The region conserved among gram-positive species is of particular interest in that it had two or more nucleotide mismatches with all of the gram-negative species (Table 2 and FIG. 4). After verifying that it fit our criteria for probe design, both regions were aligned on the 16S rRNA gene from E. coli O104:H4 and named according to the nucleotide number at which the most 5′ end of the probe was homologous: universal probe P16S683U and gram-positive probe P16S706P.

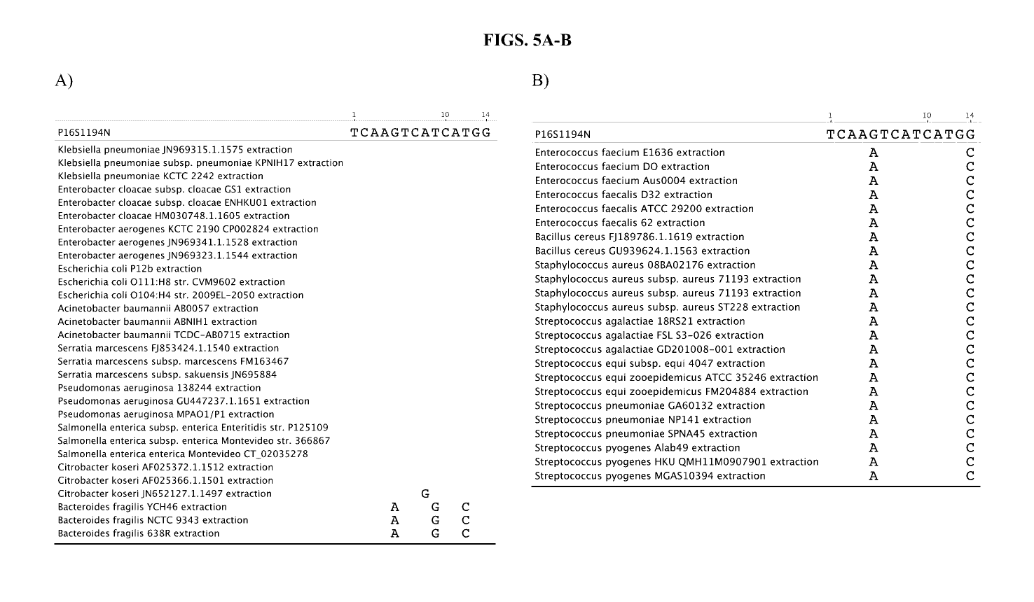

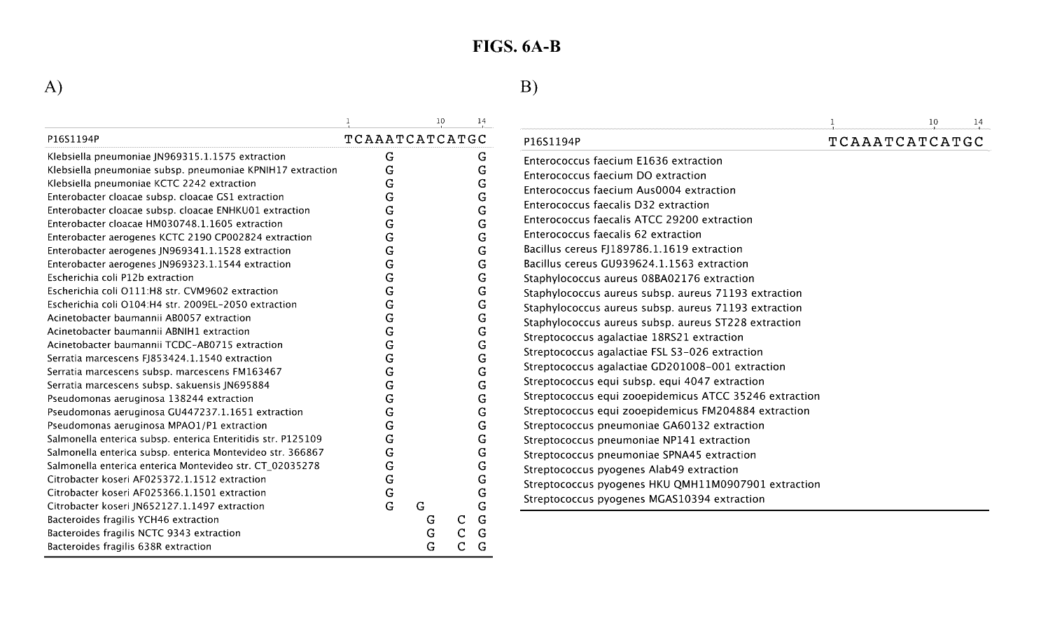

The next region identified was of particular interest because it maintained a high degree of homology among all of the species, except that there were two nucleotide mismatches between all of the gram-positive and gram-negative species, one of which was located at the 3′ end. Mismatches on the 3′ end have been shown to have the greatest discriminatory capabilities. This region was aligned on the 16S rRNA gene from E. coli O104:H4 and the probe with nearly 100% homology to the gram-negative species was named P161194N. The probe with 100% homology to the gram-positive species was named P161194P (Table 2 and FIGS. 5 and 6).

TABLE 2
Early Molecular Detection Assay Probe Sequences.
Probe sequences are listed for the upsteam ampli-
con and downstream amplicon. P16S683U is a uni-
versal probe that should detect all bacterial 16S
rRNA genes. P16S706P, P16S1194P and P16S1195P are
gram-postive-specific probes. P16S1194N and
P16S1195N are gram-negative species-specific
probes. All probes have a 5′ fluorophore
(6FAM ™, NED ™ or VIC ®) and a 3′ minor-groove
binding motif and non-fluoresent quencher
(MGHNFQ).

| | | SEQ |
|---|---|---|
| | | ID |
| | Probe Sequence (5′-3′) | NO: |
| Upstream | | |
| Probes | | |
| P16S683U | 6FAM-TTTCACCGCTACAC-MGBNFQ | 1 |
| P16S706P | NED-ATATGGAGGAACACC-MGBNFQ | 2 |
| Pl6S706P | VIC-ATATGGAGGAACACC-MGBNFQ | 3 |
| Downstream | | |
| Probes | | |
| P16S1194N | 6FAM-TCAAGTCATCATGG-MGBNFQ | 4 |
| P16S1194P | NED-TCAAATCATCATGC-MGBNFQ | 5 |
| P16S1195N | 6FAM-CAAGTCATCATGGC-MGBNFQ | 6 |
| P16S1195P | VIC-CAAATCATCATGCC-MGBNFQ | 7 |

Primer Design

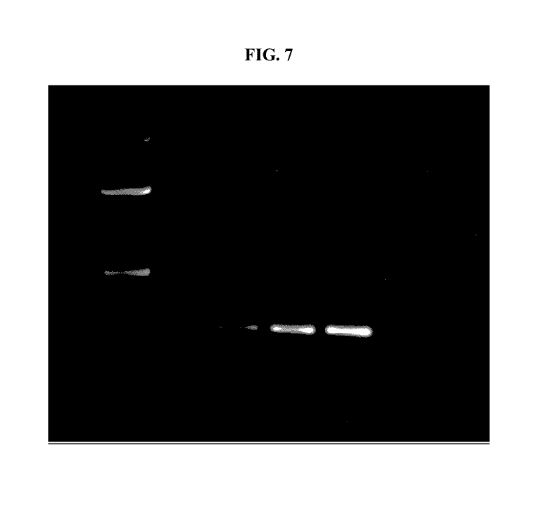

The 100 bp regions flanking the probe target regions were evaluated for primer selection. The criteria for suitable primers were sequences of 18-24 bp, with a Tₘ near 60° C. that would produce an amplicon 100 to 250 bp, with acceptable 3′ stability and minimal nucleotide repeats and single nucleotide repetitive runs. Two potential forward and two potential reverse primers were identified for the upstream probes (Table 3). The four primers designed for the upstream amplicon were tested in all possible configurations and analyzed by electrophoresis gel for amplification (FIG. 7). Three potential forward and four potential reverse primers were identified for the downstream probes. The seven primers designed for the downstream amplicon were tested in all possible configurations and analyzed by electrophoresis gel for amplification. The primers 15S557(19)F and 16S786(23)R had the best amplification efficiency and were selected for amplification of upstream amplicon. The primers 16S945(20)F and 16S1222(20)R had the best amplification efficiency and were selected for amplification of downstream amplicon.

TABLE 3
Primers tested for the amplification efficiency
for Early Molecular Detection Assay. The
primers are listed according to their target,
upstream and downstream. *Primers that were
selected for use in the EMD assay.

| | | SEQ |
|---|---|---|
| | | ID |
| | Primer Sequence (5′-3′) | NO: |
| Upstream | | |
| Primer | | |
| 16S557(19)F* | GGAATTACTGGGCGTAAAG | 8 |
| 16S560(18)F | TTTATTGGGCGTAAAGCG | 9 |
| 16S786(18)R | CTACCAGGGTATCTAATC | 10 |
| 16S786(23)R* | GTGGACTACCAGGGTATCTAATC | 11 |
| Downstream | | |
| Primer | | |
| 16S945(20)F* | GAGCATGTGGTTTAATTCGA | 12 |
| 16S946(18)F | AGCATGTGGTTTAATTCG | 13 |
| 16S968(18)F | AACGCGAAGAACCTTACC | 14 |
| 16S1191(18)R | GGCATGATGATTTGACGT | 15 |
| 16S1209(19)R | TGTAGCCCAGGTCATAAGG | 16 |
| 16S1210(18)R | TGTAGCCCAGGTCATAAG | 17 |
| 16S1222(20)R* | CATTGTAGCACGTGTGTAGC | 18 |

Primer and Probe Optimization

Compatible primer pairs were tested in conventional PCR analysis using the Qiagen QuantiFast SYBR Green PCR Kit (QIAGEN Valencia, Calif.) master mix and the amount and specificity of amplification was compared by electrophoresis through agarose. The primer pairs showing the best amplification were subsequently tested with qPCR standards, (2.85 ng/ml of S. aureus DNA, 2.81 ng/ml E. faecalis DNA, 4.96 ng/ml P. aeruginosa DNA and 5.59 ng/ml E. coli DNA), to determine the primers with universal specificity and efficiency. Primer pair 16S557(19)F/16S786(23)R was used to test the upstream probe set and primer pair R16S945(20)F/16S1222(20)R was used to test the downstream probe set in reactions with the QuantiFast Probe PCR+ROX Vial Kit. These tests were performed using, 1:10 dilutions of qPCR standards described above (S. aureus, E. faecalis, P. aeruginosa and E. coli DNA), to check for specificity and efficiency. The assay was then tested for efficiency after EMA pretreatment.

SYBR Green qPCR Analysis of Primers

The primers that gave the best results on standard PCR with electrophoresis analysis were then tested for efficiency using SYBR Green qPCR analysis. The Cₜ values were plotted against the log dilutions of DNA concentrations to create standard curves. Melting curve analysis was also preformed to test for the amplification of non-specific products. The slope of the trend line that is generated from the standard curve can determine the amplification efficiency. A trend line slope of 3.33 is indicative of 100% efficiency in the amplification cycles.

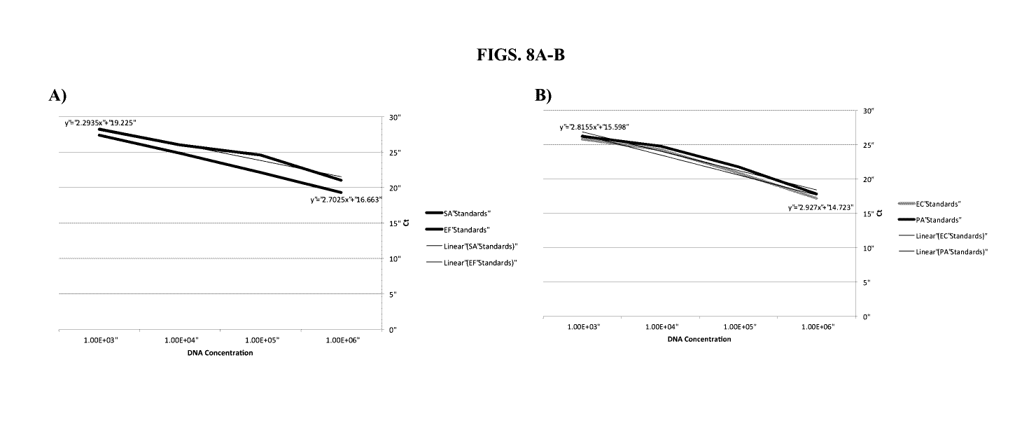

For the upstream primer set, 15S557(19)F and 16S786(23)R, a standard curve slope of 2.29 was produced on E. faecalis, 2.7 for S. aureus, 2.8 for P. aurginosa, and 2.9 for E. coli (FIG. 8). Each reaction preformed at greater than 100% efficiency, which is most likely the result of high primer concentration and slight amplification of bacterial DNA present in the qPCR master mix and reagents. This leads to a slight leveling off at the higher Cₜ values. The melting curve analysis for the upstream primers detected no amplification of non-specific products.

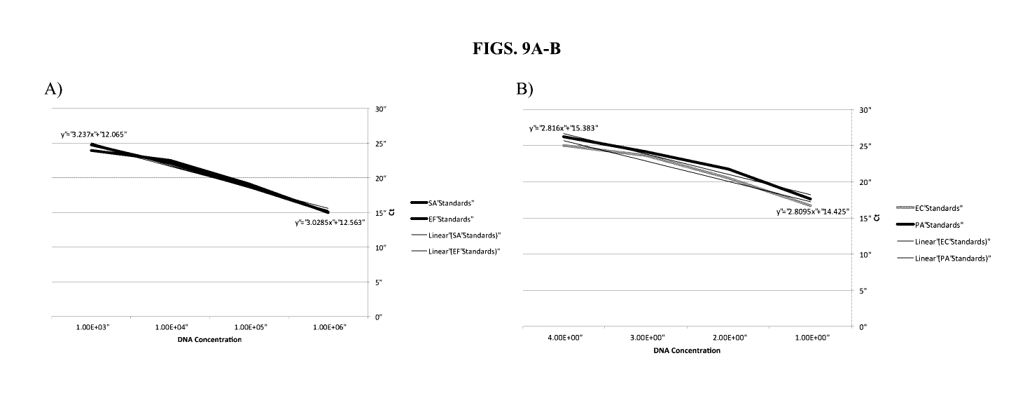

The downstream primer set, 16S945(20)F and 16S1222(20)R, produced standard curve slopes of 3 of E. faecalis, 3.2 for S. aureus, 2.8 for P. aurginosa, and 2.8 for E. coli (FIG. 9). Amplification efficiencies of greater than 100% were produced again and were likely to be a result of the same issues. Melting curve analysis showed that there were no non-specific products amplified in the reaction.

Additional optimization was performed for the assay in the Taqman configuration, so the primers with high amplification efficiency and no non-specific amplification were chosen for further assay development. No additional SYBR Green optimization was performed.

Testing Probes on Gram-Positive and Gram-Negative Genomic DNA

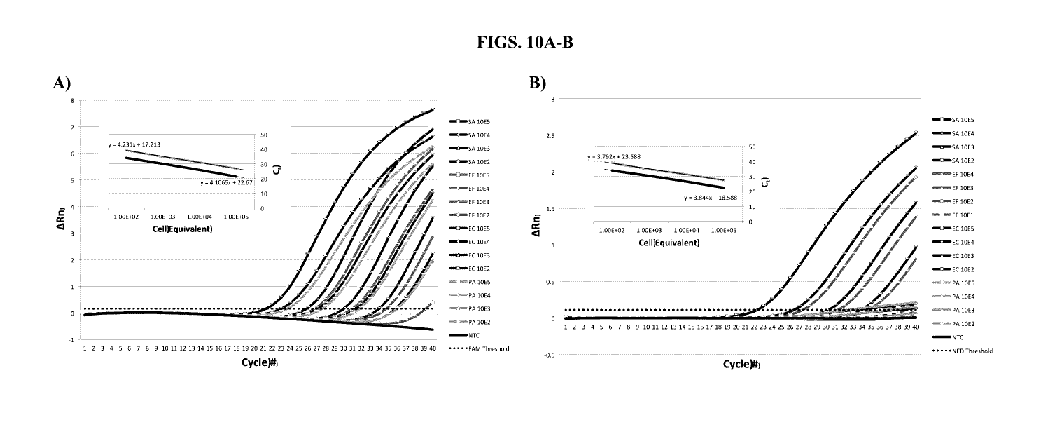

The Gram-status specificity and efficiency of the upstream and downstream primer and probe sets were determined by using as template a four order of magnitude range of genomic DNA equivalent to 10⁵ to 10² cells/ml from S. aureus, E. faecalis, P. aeruginosa and E. coli DNA. The upstream universal probe P16S683U was tested using primers 16S557(19)F and 16S786(23)R. Probe P16S683U detected all bacterial DNA tested with amplification efficiencies of approximately 75% (FIG. 10, panel A). The gram-positive probe P16S706P was tested using primers 16S557(19)F and 16S786(23)R. Probe P16S706P detected only gram-positive DNA with amplification efficiencies of approximately 86% (FIG. 10, panel B).

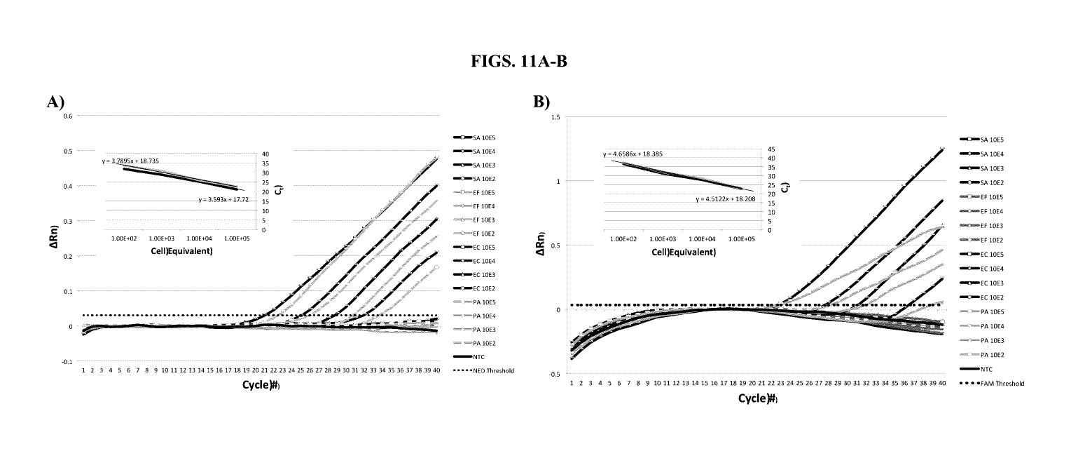

The gram-positive probe P16S1194P was tested using primers 16S945(20)F and 16S1222(20)R. Probe P16S1194P amplified the gram-positive bacterial DNA with approximately 890/% efficiency (FIG. 11, panel A). The gram-negative probe P16S1194N was testing using primers 16S945(20)F and 16S1222(20)R. Probe P16S1194N amplified the gram-negative bacterial DNA with approximately 70% efficiency (FIG. 11, panel B).

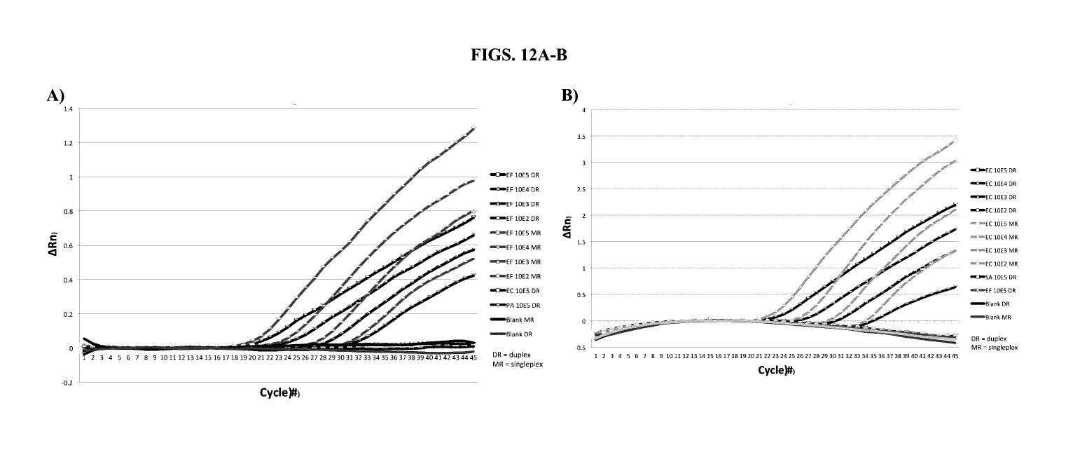

The downstream probes, P16S1194P and P16S1194N, share the same probe target region which contains two nucleotide mismatches. This could result in competition for the binding sites. An analysis of competitive binding was performed using probes P16S1194P and P16S1194N in singleplex and duplex reactions. P16S1194P was tested by using as template a four order of magnitude range of genomic DNA equivalent to 10⁵ to 10² cells/ml from E. faecalis. There was no significant difference between the specificity and efficiency in the singleplex reactions and the duplex reactions in which it was coupled with probe P16S1194N (FIG. 12, panel A). In a converse way, P16S1194N was tested by using as template a four order of magnitude range of genomic DNA equivalent to 10⁵ to 10² cells/ml from E. coli. There was no significant difference in the specificity and efficiency between the singleplex reactions and the duplexed reactions in which it was coupled with probe P16S1194P (FIG. 12, panel B).

EMA Decontamination Treatment

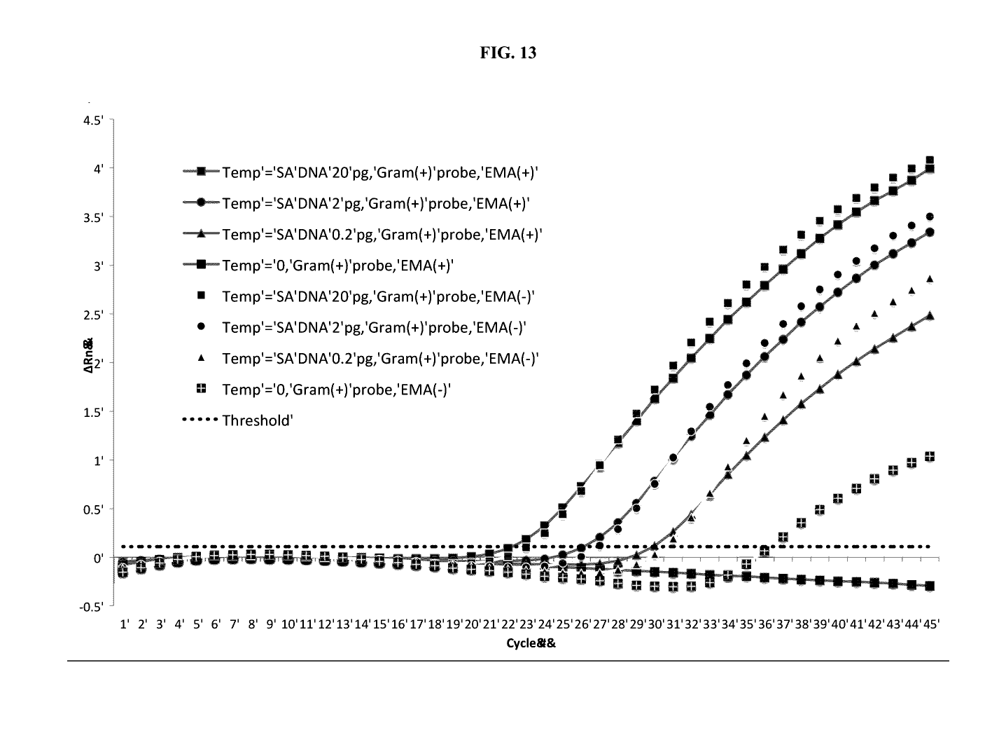

Bacterial DNA is commonly found as a contaminant in commercially available PCR and qPCR solutions and as a result is detected with probes specific for the 16S rRNA genes. To eliminate this background contamination from the reagents, the inventors treated them with EMA, which covalently intercalates into the contaminating double-stranded DNA and prevents it from further denaturation, so that it cannot serve as a template in PCR reactions. As a result, the optimization of the assay included testing pretreatment of the reagents with various concentrations of EMA (4 μM to 15 μM). At a concentration of 9 μM EMA no inhibition of the qPCR reaction was observed and the background amplification was removed (FIG. 13). In this test reaction S. aureus DNA was used at concentrations corresponding to 10⁵ (20 pg), 10⁴ (2 pg) and 10³ (0.2 pg) chromosomal equivalents. Samples were either treated or not treated with EMA (FIG. 13). Elimination of the high Ct value background amplification is observed with a 9 μM EMA pretreatment.

Synthesis and Testing of Phosphorothioate Blocking Oligonucleotides

There is the possible concern that in a multiplex reaction in which all four probes were present there would be interference from the amplification from outlying primers that occur because of the close proximity of the primers and probes (FIG. 14). To prevent this, phosphorothioate blocking oligonucleotides were made to prevent the generation of large PCR amplicons that arise from the amplification of products from outlying primers that occur in multiplex PCR reactions with targets in close proximity (FIG. 14).

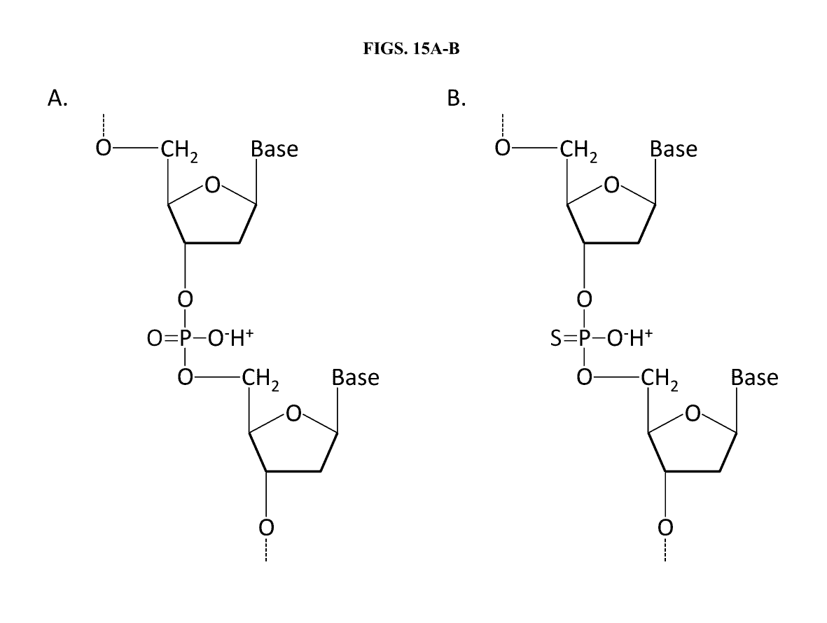

Phosphorothioates are oligonucleotides in which a sulfur atom replaces one of the non-bridging oxygen's on the phosphate linkage (FIG. 15, panel B). This modification may provide greater resistance to hydrolysis including to the exonucleolytic activity of DNA polymerase.

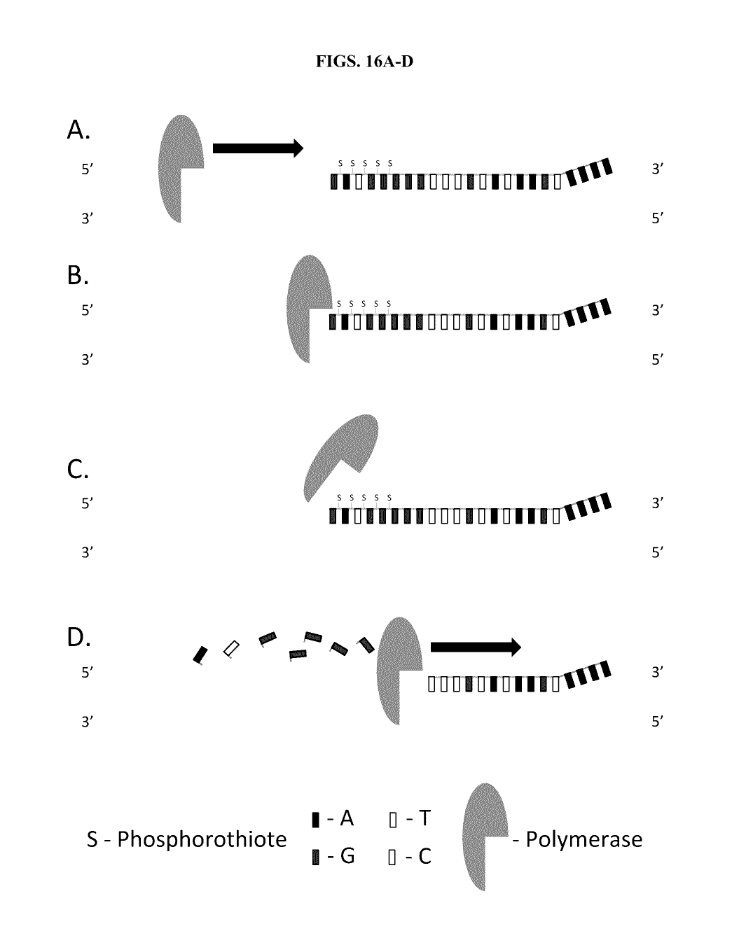

The inventors designed a functional phosphorothioate containing oligonucleotide designated a ‘blocking oligo’ that anneals to the template DNA between the reverse primer of an upstream target (UpRP) and the forward primer of the downstream product (DnRP) and prevents the amplification of the entire undesired amplicon (UpFP to DnRP) (FIG. 16). Specifically, a 23-mer ‘blocking oligo’ that contained five phosphorothioate-modified linkages at the 5′ end that are directly followed by a nine nucleotide region enriched in guanine and cytosine nucleotides, termed a ‘GC clamp’, and includes four adenines at the 3′ end was designed. The phosphorothioate modifications block the 5′ to 3′ exonuclease activity of the DNA polymerase and terminate extension. The ‘GC clamp’ ensures specific and strong annealing. The four 3′ adenines are designed to be mismatches and prevent possible extension from the blocking oligonucleotide. These specific design elements will be different for each target, but will usually include four to five phosphorothioate-modified linkages at the 5′ end, followed by a strong ‘GC clamp’, and have four to five mismatches at the 3′ end.

The examples herein illustrate that phosphorothioates can be used as a barrier to PCR amplification. Experimental results show that a primer containing all of our blocking oligo parameters except phosphorothioate modifications is not capable of blocking flanking amplification. However, this same nucleotide sequence with the phosphorothioate modifications is able to block flanking amplification (FIG. 17). It is anticipated that these ‘blocking oligos’ will have important applications.

### Example 3

### S. aureus Viability with EMA and PMA Exposure

S. aureus cells exposed to EMA-treated PBS for 10 and 30 min and to non-EMA treated PBS as a control for a 10 min incubation identified an issue with EMA-treated PBS and an approximately a log loss in cell viability when compared to the non-EMA exposed cells (see, e.g., Table 4).

Furthermore, after exposure to EMA-treated PBS for 30 min no viable cells were recovered. To obviate this negative effect of EMA on cell viability, it was determined that by replacing EMA with PMA there was no significant difference in cell viability following treatment with PMA-treated PBS and the non-treated PBS. EMA treatment was therefore replaced with PMA for use in the reagent pretreatment protocol.

### Example 4

### qPCR Assay in Artificial Blood Samples

Detection of S. aureus DNA in Blood and PBS

To establish the sensitivity and reproducibility of the EMD assay, artificial blood samples were prepared containing each of four laboratory strains of representative gram-positive (S. aureus and E. fecalis) and gram-negative (E. coli and P. aeruginosa) bacteria at concentrations ranging from 10⁵ to 10¹ cell/ml. As an additional control, similar samples were prepared in PBS. Each of these eight samples was treated using the standard EMD assay protocol. The DNA was extracted, quantified, and a qPCR reaction was performed and analyzed. The efficiency of the whole protocol from extraction through EMD analysis was compared to the input cell concentration that is based on colony counts.

The S. aureus DNA detected in the blood ranged from 34 to 83 percent of the number of input cells from which it was extracted (2.3×10⁵ to 2.3×10¹ cells/ml). The limit of detection of S. aureus cells was estimated at 100 cells/ml, as the DNA was not always detected in blood at input cell concentrations less than 10² cells/ml. As described above, the DNA detected from the cells resuspended in EMA-treated PBS had a poor correlation with the input cell concentrations. The S. aureus DNA detected in the EMA-PBS ranged from 0 to 20 percent of the numbers of input cells from which it was extracted. The universal and gram-positive probes amplified the DNA samples and no signal was detected from the gram-negative probe (Table 4).

TABLE 4
Detection of S. aureus DNA from inoculated blood samples.

| CFU per ml | | | | | | |
|---|---|---|---|---|---|---|
| added to | | | | | | |
| blood | | | | | | |
| (determined | | | | | | |
| by plate | Upstream | | Downstream | | | |
| count) | Universal | Gram+ | Gram+ | Gram− | Average value | % Detection |
| | Blood | | | | | |
| 2.3E5 | 103840 (45%) | 64620 (28%) | 68260 (29%) | — | 7.8 × 10⁴ | 34 |
| 2.3E4 | 13790 (60%) | 13870 (60%) | 15720 (68%) | — | 1.4 × 10⁴ | 61 |
| 2.3E3 | 1290 (56%) | 2260 (98%) | 2160 (94%) | — | 1.9 × 10³ | 83 |
| 2.3E2 | 150 (65%) | 80 (35%) | 50 (22%) | — | 9.3 × 10¹ | 40 |
| 2.3E1 | 60 (260%) | — | — | — | — | — |
| | PBS (EMA-treated) | | | | | |
| 2.3E5 | 6180 (3%) | 4370 (2%) | 18230 (8%) | — | 9.5 × 10³ | 4 |
| 2.3E4 | 570 (2%) | 340 (1%) | 3730 (16%) | — | 4.6 × 10³ | 20 |
| 2.3E3 | 2 (1%) | — | 260 (11%) | — | — | — |
| 2.3E2 | — | — | — | — | — | — |
| 2.3E1 | — | — | — | — | — | — |

Detection of E. faecalis DNA in Blood and PBS

The E. faecalis DNA detected in the blood ranged from 34 to 81 percent of the number of input cells from which it was extracted. The limit of detection of E. faecalis cells, similar to S. aureus, was estimated at 100 cells/ml, as often no DNA was detected in the blood at concentrations less than 10² cells/ml. In this case, the DNA extracted from cells resuspended in PBS has more correlation with the number of input cells. The E. faecalis DNA detected in PBS ranged from 60 to 132 percent of the numbers of input cells. The universal and gram-positive probes amplified the DNA samples and no signal was detected from the gram-negative probe (Table 5).

TABLE 5
Detection of E. faecalis DNA from inoculated blood samples.

| CFU per ml | | | | | | |
|---|---|---|---|---|---|---|
| added to | | | | | | |
| blood | | | | | | |
| (determined | | | | | | |
| by plate | Upstream | | Downstream | | | |
| count) | Universal | Gram+ | Gram+ | Gram− | Average value | % Detection |
| | Blood | | | | | |
| 3.6E5 | 208080 (57%) | 113270 (31%) | 50940 (14%) | — | 1.2 × 10⁵ | 34 |
| 3.6E4 | 19510 (54%) | 1735 (48%) | 30880 (85%) | — | 2.2 × 10⁴ | 63 |
| 3.6E3 | 1630 (45%) | 2110 (58%) | 2930 (81%) | — | 2.2 × 10³ | 62 |
| 3.6E2 | 420 (116%) | 170 (47%) | 280 (77%) | — | 2.9 × 10² | 81 |
| 3.6E1 | — | — | — | — | — | — |
| | PBS | | | | | |
| 3.6E5 | 384050 (106%) | 196720 (54%) | 64650 (17%) | — | 2.1 × 10⁵ | 60 |
| 3.6E4 | 41700 (115%) | 29920 (83%) | 40480 (112%) | — | 3.7 × 10⁴ | 103 |
| 3.6E3 | 4510 (125%) | 4710 (130%) | 5090 (141%) | — | 4.7 × 10³ | 132 |
| 3.6E2 | 350 (97%) | 420 (116%) | 190 (52%) | — | 3.2 × 10² | 89 |
| 3.6E1 | 40 (111%) | — | — | — | — | — |

Detection of E. coli DNA in Blood and PBS

The E. coli DNA detected in the blood ranged from 66 to 169 percent of the numbers of input cells from which it was extracted. The limit of detection of E. coli cells in blood was approximately 10 cells/ml with the upstream universal probe, as DNA was detected at all the DNA concentrations. The limit of detection of was approximately 100 cells/ml with the downstream gram-negative probe, as DNA was often not detected in the blood at cell concentrations less than 10² cells/ml. The E. coli DNA detected in PBS ranged from 70 to 198 percent of the number of input cells. The universal and gram-negative probes amplified the DNA samples and no signal was detected using the gram-positive probes (Table 6).

TABLE 6
Detection of E. coli DNA from inoculated blood samples.

| CFU per ml | | | | | | |
|---|---|---|---|---|---|---|
| added to | | | | | | |
| blood | | | | | | |
| (determined | | | | | | |
| by plate | Upstream | | Downstream | | | |
| count) | Universal | Gram+ | Gram+ | Gram− | Average value | % Detection |
| | Blood | | | | | |
| 3.6E5 | 281360 (78%) | — | — | 194680 (54%) | 2.3 × 10⁵ | 66 |
| 3.6E4 | 27890 (77%) | — | — | 62370 (173%) | 4.5 × 10⁴ | 125 |
| 3.6E3 | 2590 (71%) | — | — | 9610 (266%) | 6.1 × 10³ | 169 |
| 3.6E2 | 410 (113%) | — | — | 402 (111%) | 4 × 10² | 112 |
| 3.6E1 | 180 (692%) | — | — | — | — | — |
| | PBS | | | | | |
| 3.6E5 | 209440 (81%) | — | — | 155770 (60%) | 1.8 × 10⁵ | 70 |
| 3.6E4 | 9740 (38%) | — | — | 29240 (113%) | 3.8 × 10⁴ | 75 |
| 3.6E3 | 1020 (40%) | — | — | 6910 (265%) | 3.9 × 10³ | 152 |
| 3.6E2 | 170 (65%) | — | — | 860 (330%) | 5.1 × 10² | 198 |
| 3.6E1 | — | — | — | — | — | — |

Detection of P. aeruginosa DNA in Blood and PBS

The P. aeruginosa DNA detected in the blood ranged from 84 to 225 percent of the number of input cells. The limit of detection of P. aeruginosa cells was approximately 10 cells/ml with the upstream universal probe, as DNA was detected in samples at all DNA concentrations. The limit of detection of P. aeruginosa cells was approximately 100 cells/ml with the downstream gram-negative probe, as DNA was often not detected in the samples at cell concentrations less than 10² cells/ml. The P. aeruginosa DNA detected in PBS ranged from 74 to 140 percent of the number of input cells. The limit of detection for the PBS was approximately 10 cells/mi. The universal and gram-negative probes amplified the DNA samples and no signal was detected from the gram-positive probes (Table 7).

TABLE 7
Detection of P. aeruginosa DNA from inoculated blood samples.

| CFU per ml | | | | | | |
|---|---|---|---|---|---|---|
| added to | | | | | | |
| blood | | | | | | |
| (determined | | | | | | |
| by plate | Upstream | | Downstream | | | |
| count) | Universal | Gram+ | Gram+ | Gram− | Average value | % Detection |
| | Blood | | | | | |
| 4.6E5 | 568840 (123%) | — | — | 212810 (46%) | 3.9 × 10⁵ | 84 |
| 4.6E4 | 77370 (168%) | — | — | 23630 (51%) | 5 × 10⁴ | 109 |
| 4.6E3 | 8430 (183%) | — | — | 7890 (171%) | 8.1 × 10³ | 177 |
| 4.6E2 | 1070 (232%) | — | — | 1016 (220%) | 1 × 10³ | 225 |
| 4.6E1 | 60 (130%) | — | — | — | — | — |
| | PBS | | | | | |
| 4.6E5 | 413170 (89%) | — | — | 297490 (114%) | 3.5 × 10⁵ | 77 |
| 4.6E4 | 44680 (97%) | — | — | 84550 (183%) | 6.4 × 10⁴ | 140 |
| 4.6E3 | 4720 (102%) | — | — | 2116 (46%) | 3.4 × 10³ | 74 |
| 4.6E2 | 820 (178%) | — | — | 264 (57%) | 5.4 × 10² | 117 |
| 4.6E1 | 14 (30%) | — | — | 70 (152%) | 4.2 × 10¹ | 91 |

### Example 5

### Analysis of Clinical Samples

Eighteen clinical samples were simultaneously analyzed using the EMD assay and by the Memorial Hermann Hospital Clinical Microbiology Laboratory. The Clinical Laboratory, using culture and Gram-stain analysis classified two of the 18 clinical samples (11%) as positive and 16 (89%) as negative (Table 8). Of the two positive samples, both were identified as gram-negative by both the Clinical Microbiology Laboratory and the EMD assay. This represents 100% congruence between the samples that were measured as positive by the ‘gold standard’ of blood culture and Gram stain. Of the 16 culture negative samples six were also determined by the EMD assay to lack amplifiable bacterial DNA above a threshold equivalent to 100 cells/ml. This represents 37% congruence between the culture-negative samples and the EMD assay negative samples. Ten of the 16 culture negative samples resulted in amplification of bacterial DNA above a 100-cell/ml threshold. This represents 62% non-congruence between the culture-negative samples and the EMD assay. The average time to determination of a positive blood culture result was 20 hr (Table 8). The TTD for the EMD assay was estimated at 4.5 hr by calculating the times required for sample processing, DNA extraction, and qPCR analysis.

The plasma DNA was extracted and samples #8 through 18 were used as a template for the EMD assay (Table 9). For two of these samples (#8 and 16) there was no amplification above background detected for the plasma or the pelleted samples. For five of the samples (#11, 12, 13, 17, and 19) the plasma values were negligible, whereas the pellet samples were above background. For three of these samples (#9, 10, and 14) the plasma values were above background and ranged greatly (18 to 91% of the pelleted value), but were always less than the pellet levels. For all of the samples, except #18, the values for the universal primer were negligible. However, #18 was only amplified by the universal primer for both the plasma and pellet samples and the plasma amplification was 27% of that of the pellet. For only one sample (#15) that was positive by both culture and Gram strain and EMD assay, was the value in the plasma more than the pellet. In this case the amplification in plasma indicated that there was greater than 100 times more DNA in the plasma than in the pellet. This patient was released from the hospital, so presumably the antibiotic treatment was functioning and caused many lysed cells to be present in the plasma.

TABLE 8
Analysis of clinical blood samples with the Early Molecular
Detection assay.

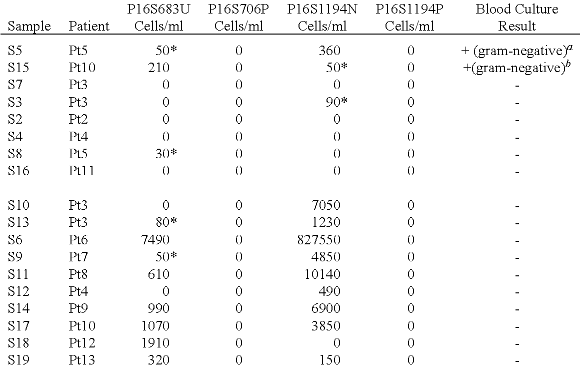

The first two samples are classified as positive by blood culture and the remaining samples were negative by blood culture.
All data above the gray bar include samples where there is 100% agreement between EMD assay and blood culture results. Data below the gray bar are samples that resulted in incongruent results (positive EMD assay but negative blood culture results).
The asterisk indicates assay results below the limit of detection.
The values (cell/ml) are calculated based on E. coli standards for the samples that were detected by the universal and Gram-negative probes.
^{a}22 hr to blood culture results.
^{b}18 hr to blood culture results.

TABLE 9
Analysis of clinical blood sample pellet compared with plasma.
Pellets and plasma were analyzed for each sample. The plasma, samples are labeled with
“P” at the end of the sample name. The asterisk indicates assay results below
the limit of detection. The values (cell/ml) are calculated based on E. coli
standards for the samples that were detected by the universal and Gram negative probes.

| | | P16S683U | P16S706P | P16S1194N | P16S1194P | |
|---|---|---|---|---|---|---|
| Sample | Patient | Cells/ml | Cells/ml | Cells/ml | Cells/ml | BC Result |
| S8 | Pt5 | 30 | 0 | 0 | 0 | − |
| S8P | Pt5 | 0 | 0 | 10* | 20* | − |
| S16 | Pt11 | 0 | 0 | 0 | 0 | − |
| S16P | Pt11 | 0 | 0 | 0 | 0 | − |
| S12 | Pt4 | 0 | 0 | 490 | 0 | − |
| S12P | Pt4 | 0 | 0 | 0 | 0 | − |
| S17 | Pt10 | 1070 | 0 | 3850 | 0 | − |
| S17P | Pt10 | 0 | 0 | 0 | 0 | − |
| S19 | Pt13 | 320 | 0 | 150 | 0 | − |
| S19P | Pt13 | 0 | 0 | 0 | 0 | − |
| S11 | Pt8 | 610 | 0 | 10140 | 0 | − |
| S11P | Pt8 | 0 | 0 | 90* | 0 | − |
| S13 | Pt3 | 80 | 0 | 1230 | 0 | − |
| S13P | Pt3 | 0 | 0 | 17* | 0 | − |
| S9 | Pt7 | 50 | 0 | 4850 | 0 | − |
| S9P | Pt7 | 20 | 0 | 880 (55%) | 0 | − |
| S14 | Pt9 | 990 | 0 | 6900 | 0 | − |
| S14P | Pt9 | 0 | 0 | 3830 (18%) | 0 | |
| S10 | Pt3 | 0 | 0 | 7050 | 0 | − |
| S10P | Pt3 | 0 | 0 | 6450 (91%) | 0 | − |
| S18 | Pt12 | 1910 | 0 | 0 | 0 | − |
| S18P | Pt12 | 510 (27%) | 0 | 0 | 0 | − |
| S15 | Pt10 | 210 | 0 | 50* | 0 | +(gram-negative)^{a} |
| S15P | Pt10 | 0 | 0 | 39190 | 0 | +(gram-negative)^{a} - |
| | | | | (>100%) | | |

^{a}18 hr to blood culture results.

### Example 6

### Early Molecular Detection Assay Protocol

Bacteria Culture for Standards in Blood

Bacterial stocks were made by mixing 300 μl of 50% glycerol and 700 μl of overnight culture in a labeled cryovial. The stocks were then immediately stored in a −80° C. freezer. On day 1 of the assay, a portion of the frozen bacteria stock was plated on either tryptic soy agar (TSA) (S. aureus), brain heart infusion (BHI) agar (E. faecalis) or Luria Bertani (LB) agar (E. coli and P. aeruginosa) using a sterile wooden stick or a sterile loop to obtain isolated colonies. The plates were then incubated at 37° C. overnight to allow the bacterial colonies to grow. At the end of day 2, 5 ml of the appropriate liquid media was inoculated in a 15×100 mm test tube with 3 colonies from the fresh agar plate as described above. The inoculated tubes were incubated at 37° C. with shaking overnight.

On day 3, subcultures were generated by placing 50 μl of the overnight culture in 5 ml of the appropriate liquid media in a 15×100 mm test tube. The inoculated tubes were again incubated at 37° C. with shaking. The optical density of the subculture was measured until the OD_{600 nm} was approximately 0.5, which took about two to four hours. To measure the OD_{600 nm} of the solution, the spectrophotometer was blanked with 1 ml of the same liquid media in a clean cuvette. Then 1 ml of the subculture was added into a clean cuvette and the OD_{600 nm} was measured.

The cells were harvested by centrifugation and the pellet was resuspended in PBS, such that the OD_{600 nm} of the solution was 0.5 in 500 μl PBS (500 μl of OD_{600 nm}=0.500 bacterial solution). First, the amount of subculture needed to get the OD_{600 nm}=0.500 was calculated using the formula C1V1=C2V2; V1=C2V2/C1. For example: V1=0.457 ml, when C1=0.547 [0.5×0.5/0.547]. Then the appropriate amount of the culture was transferred into a sterile 1.5 ml microcentrifuge tube. After centrifugation and removal of the supernatant fluid, the bacterial pellet was resuspended into 0.5 ml of PMA-treated PBS. Subcultures for various bacteria were made as shown in Table 10 below.

TABLE 10
Bacteria subcultures.

| | |
|---|---|
| OD₆₀₀ = 0.500 of E. coli = 4 × 10⁸ CFU/ml | OD₆₀₀ = 0.500 of E. faecalis = 5 × 10⁸ CFU/ml |
| 12.5 ul cells + 487.5 ul PMA-PBS ~1.0 × 10⁷ CFU/ml | 10 ul cells + 490 ul PMA-PBS ~1.0 × 10⁷ CFU/ml |
| OD₆₀₀ = 0.500 of P. aeruginosa = 1.0 × 10⁸ CFU/ml | OD₆₀₀ = 0.500 of S. aureus = 1 × 10⁸ CFU/ml |
| 50 ul cells + 450 ul PMA-PBS ~1.0 × 10⁷ CFU/ml | 50 ul cells + 450 ul PMA-PBS ~1.0 × 10⁷ CFU/ml |

### Addition of Bacterial Dilutions to Blood Samples

The OD₆₀₀=0.500 solution was diluted to a concentration of ˜1×10⁷ CFU/ml using a known concentration of the bacteria and C₁V₁=C₂V₂. (See Table 10 above.) A 1×10⁶ CFU/ml solution was generated by adding 50 μl of the 1×10⁷ CFU/ml solution to 450 μl of PMA-PBS in a sterile 1.5 ml microcentrifuge tube. Five more serial 1/10 dilutions were generated from the 1×10⁶ CFU/ml solution (1×10⁵ CFU/ml to 1×10¹ CFU/ml). To determine if there was any bacteria naturally present in the blood sample, 100 μl of blood was saved and 50 μl of blood was placed onto each of two room temperature blood agar plates (plates had been stored at 4° C.). The blood was spread on the surface of the agar with a sterile glass spreader. One plate was incubated at 37° C. and the other plate was immediately incubated at 37° C. in an anaerobic chamber.

The remaining blood was distributed in 1 ml increments into labeled 2-ml MO BIO collection tubes. Ten μl of the 1×10⁶ CFU/ml solution was added into 1 ml of blood and vortexed well to mix (NOTE: 10 μl of 1×10⁶ CFU/ml is 1×10⁴ CFU added to the 1 ml blood sample). This process was repeated for dilutions 1×10⁵ CFU/ml, 1×10⁴ CFU/ml, and 1×10³ CFU/ml. All of the blood samples were centrifuged for five minutes at 13,000×g. The serum was removed from each tube and the blood pellets were stored at −20° C. or −80° C. All of the dilutions (10⁶ to 10¹) were then plated to confirm the bacterial concentrations. Each agar plate was divided into six equal wedges and three 10 μl drops of each dilution were placed into the appropriate section. The plates were incubated overnight at 37° C. The number of distinguishable colonies were counted after about 18 hours. The bacterial concentrations were calculated by averaging the three 10 μl spots to determine CFU/ml.

DNA Extraction from Blood Samples

The previously frozen blood samples were thawed. Simultaneously, a bottle of CB1 was placed on the 70° C. heating block to warm up. CB1 (450 μl) was mixed into each blood-containing tube by pipetting up and down using a P1000 and the fluid was then transferred to a 2-ml tube containing 0.1 mm glass beads (MO BIO). Beta-mercaptoethanol (BME) (47.5 ul) was added to each tube (˜5% v/v). The tubes were vortexed to loosen the glass beads at the bottom of the tubes. The tubes were then placed in the 70°-80° C. water-filled heating block for 15 min. The tubes were placed in the Mini-BeadBeater 8 (Biospec, Bartlesville, Okla.), set on ‘homogenize’ for 10 min. Afterwards the tubes were centrifuged at 10,000×g for 1 min and the supernatant was transferred into new labeled 2-ml collection tubes. PMA-treated CB2 (100 μl) was added to each tube. The tubes were vortexed briefly and left to sit at room temperature for 5 to 10 min. Then the tubes were centrifuged at 10,000×g for 1 min and the supernatants were transferred into new labeled 2-ml collection tubes, with care taken to not transfer any of the white precipitate. PMA-treated CB3 (1 ml) was added to each tube, and mixed by inversion or gentle pipetting. All of each sample (600 μl at one time) was loaded into a filter cartridge affixed to a vacuum manifold that had been washed with 75% ethanol. The filter cartridges were removed from the vacuum manifold and placed in new 2-ml collection tubes. The tubes were centrifuged at 10,000×g for 1 min and returned to the vacuum manifold. The filters cartridges were then washed twice with 500 μl PMA-treated CB4. The filters cartridges were next removed from the vacuum manifold and return to the previously used 2-ml collection tube. The tubes were centrifuged at 13,000×g for 2 min to dry the column. The filter cartridges were then transferred to new 2-ml collection tubes and 50 μl of PMA-treated CB5 was added directly to each filter membrane and left for 5 min. The filter cartridges and tubes were centrifuged at 10,000×g for 1 min and the tubes were frozen at −20° C. until use.

Quantitative PCR Reactions

Two master mixes of primers were made: (1) upstream (primers: 16S557(19)F, 16S786(23)R; probes: P16S683U, P16S706P), (2) downstream (primers: 16S945(20)F, 16S1222(20)R; probes: P16S1195N, P16S1195P). For the EMA treatment, the calculated amount of diluted 239.7 uM EMA solution (0.53 ul per 14 ul master mix) was added to each master mix in the dark room with as little light as possible and mixed well (see Table 11 below). The EMA was then allowed to bind for 5 min at room temperature. The master mix tubes were placed on ice horizontally and exposed to a 500 W light bulb placed 10 cm away for 5 min.

TABLE 11
Exemplary reagent mix

| Reagent | Amount per well | |
|---|---|---|
| PCR Master Mix | 10 | μL |
| Forward Primer | 1 | μL |
| Reverse Primer | 1 | μL |
| EMA | 0.53 | μL |
| H₂0 | 1.47 | μL |
| Probe 1 | 0.5 | μL |
| Probe 2 | 0.5 | μL |

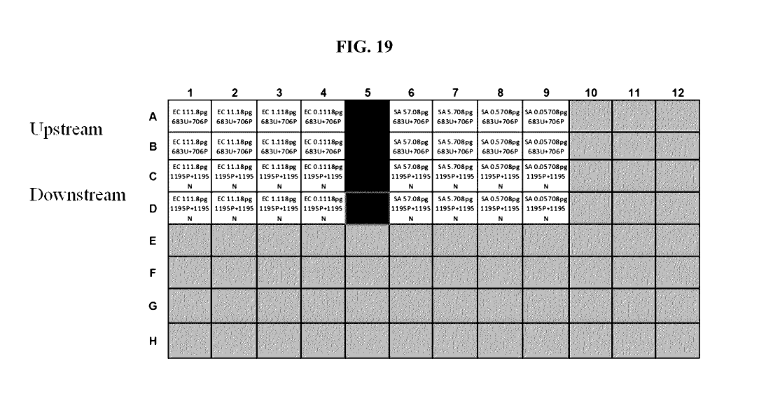

The probes were added to the EMA-treated master mixes. Master mix (15 μl) was added to each appropriate well of a 96-well plate on a plate ice block. Then 5 μl of sample (undiluted or 1/10 dilution) and DNA standards were added to their appropriate wells. The standards were then plated as shown in FIG. 19. The plate was sealed with film and centrifuged briefly.

PCR Analysis Using the BioRad qPCR Machine

The plate as described above was placed in the qPCR machine and treated according to Table 12 below.

TABLE 12
PCR Program

| | | |
|---|---|---|
| 95° C. | 3 min | |
| 95° C. | 10 sec | |
| 58° C. | 5 sec | x 45 |
| 60° C. | 1 min | |

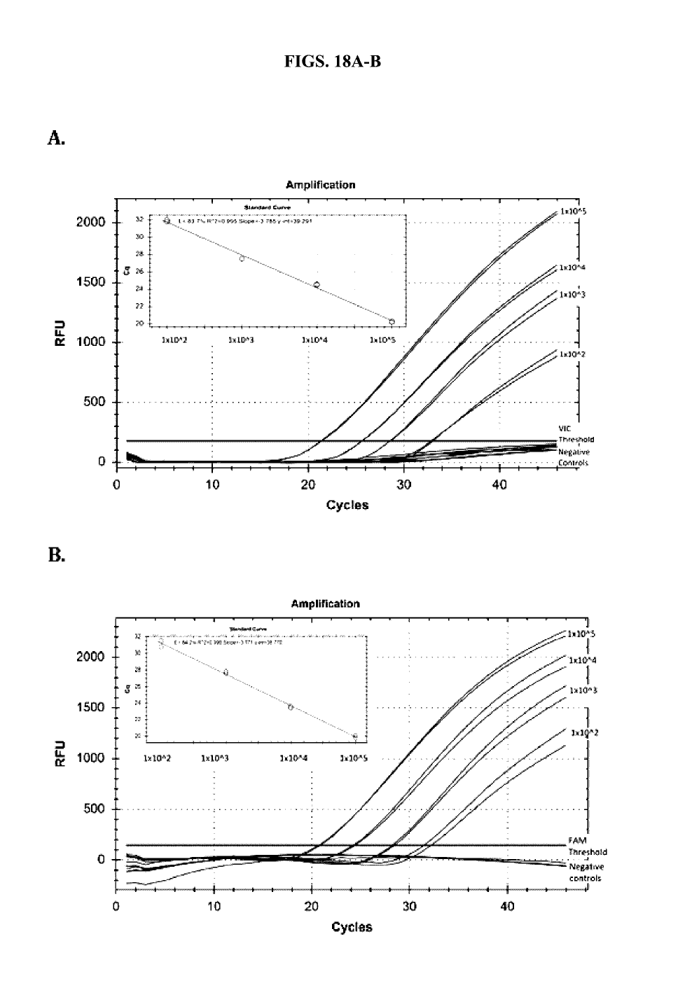

The results were analyzed for fluorescent detection and standard curve plots for the downstream amplicons using the probes P16S1195P(VIC) and P16S1195N(6FAM). FIG. 18A shows probe P16S1195P analyzing E. coli and S. aureus DNA at 1×10⁵ to 1×10² cell equivalents. All of the E. coli DNA amplification is below the threshold value. FIG. 18B shows probe P16S1195N analyzing E. coli, and S. aureus DNA at 1×10⁵ to 1×10² cell equivalents. All of the S. aureus DNA amplification is below the threshold value.

Recipes

The following recipes were used in the assay described above:
1. Primers: mixed 1:5 ratio in H₂O [Note: EMA-treated with master mix.]
2. Probes: mixed 1:10 ratio in EMA-treated H₂O
3. EMA: undiluted 23.97 mM/diluted 1/100 working stock 0.24 mM (239.7 uM)
4. PMA: undiluted 20 mM/diluted 1/100 working stock 0.2 mM (200 uM)
5. EMA-treated Master Mix (9 uM): 0.53 ul diluted EMA (239.7 uM) per 14 ul master mix

6. EMA-treated H₂O (9 uM): 0.53 ul diluted EMA (239.7 uM) per 14 ul water. This was used for template control, probe dilutions, and DNA standards.

For the PMA treatment of extraction reagents, the following concentrations were used:
1. CB1: untreated
2. CB2: 1.5 μl freezer stock PMA for each bottle (6 ml)
3. CB3: 7 μl freezer stock PMA for each bottle (28 ml)
4. CB4: 7.5 μl freezer stock PMA for each bottle (30 ml)
5. CB5: 1.5 μl freezer stock PMA for each bottle (6 ml)

To make EMA or PMA 1/100 dilutions for treatment of solutions, a sterile screw cap glass vial (4.5 ml) was wrapped completely with tape to prevent light exposure. 495 μl of dimethyl formamide (DMF) was added, followed by 5 μl of EMA or PMA in a dark room. The vials were then vortexed to mix.

Bacterial DNA Standards

To make the DNA standards used in this assay, DNA was extracted from a 500 μl bacterial overnight culture using a BiOstic Bacteremia DNA Isolation Kit (MO BIO Cat. No. 12240-50). The DNA concentrations of 1 μl portions were determined using either the plate reader or NanoDrop. The DNA was diluted initially to 1 ng/μl using the formula C₁V₁=C₂V₂ into EMA- or PMA-treated water. The DNA was then further diluted so that there was 1×10⁵ CFU/μl, 1×10⁴ CFU/μl, 1×10³ CFU/μl, and 1×10² CFU/μl dilutions of DNA using C₁V₁=C₂V₂. The concentrations of the standards were as follows: for E. coli 111.82 pg/μl=1×10⁶ CFUs/μl; for S. aureus 57.08 pg/μl=1×10⁶ CFUs/μl.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method for detecting bacteria in a biological sample from a subject comprising:
(a) amplifying at least two regions of a sequence encoding bacterial ribosomal RNA (rRNA) to generate at least two bacterial amplicons, wherein at least one of the two regions comprises sequences that are shared between gram-positive and gram-negative bacteria and sequences that are different between gram-positive and gram-negative bacteria;
(b) hybridizing each of the at least two amplicons with at least two probes to produce a detectable signature, wherein at least one of said probes that hybridizes to each of the amplicons is specific for gram-positive or gram-negative bacterial sequences and one of said probes that hybridizes to one of the amplicons is non-specific for gram-positive or gram-negative bacterial sequences; and
(c) identifying the presence of bacteria in the sample based in the detectable signature.

2. An assay method comprising:
(a) amplifying at least two regions of a sequence encoding bacterial ribosomal RNA (rRNA) from a biological sample from a subject to generate at least two bacterial amplicons, wherein at least one of the two regions comprise sequences that are shared between gram-positive and gram-negative bacteria and sequences that are different between gram-positive and gram-negative bacteria; and
(b) hybridizing each of the at least two amplicons with at least two probes to produce a detectable signature, wherein at least one of said probes that hybridizes to each of the amplicons is specific for gram-positive or gram-negative bacterial sequences and one of said probes that hybridizes to one of the amplicons is non-specific for gram-positive or gram-negative bacterial sequences.

3. The method of claim 1, wherein said amplifying comprises amplifying DNA from the bacterial genome.

4. The method of claim 1, wherein said amplifying does not involve reverse transcription.

5. The method of claim 1, further defined as a method for detecting pathogenic bacteria.

6. The method of claim 5, wherein the method detects greater than 100 bacterial cells per ml of sample.

7. The method of claim 5, wherein the method detects greater than 10 bacterial cells per ml of sample.

8. The method of claim 1, further defined as a method for detecting S. aureus, E. faecalis, P. aeruginosa and/or E. coli bacteria.

9. The method of claim 1, wherein the sample is a blood sample.

10. The method of claim 1, further defined as a method for determining whether the bacteria are gram-negative or gram-positive bacteria.

11. The method of claim 1, wherein step (c) further comprises identifying the presence of gram-positive or gram-negative bacteria in the sample.

12. The method of claim 11, further comprising reporting the presence of gram-positive or gram-negative bacteria in the sample.

13. The method of claim 12, wherein the reporting is reporting to a doctor, a hospital, an insurance company or to the subject.

14. The method of claim 12, wherein the reporting comprises preparing a written or electronic report.

15. The method of claim 1, wherein step (c) further comprises diagnosing the subject with bacterial sepsis.

16. The method of claim 1, further defined as an in vitro method.

17. The method of claim 1, wherein the method if performed in less than 6 hours.

18-44. (canceled)

45. A kit comprising:
(i) at least two primer pairs suitable for amplification of two regions of sequence encoding bacterial ribosomal RNA (rRNA), wherein at least one of the two regions comprises sequences that are shared between gram-positive and gram-negative bacteria and sequences that are different between gram-positive and gram-negative bacteria; and
(ii) at least three hybridization probes, wherein two of the probes are specific for gram-positive or gram-negative bacterial sequences and one of the probes is non-specific for gram-positive or gram-negative bacterial sequences.

46. The kit of claim 45, comprising at least four hybridization probes, wherein three of the probes are specific for gram-positive or gram-negative bacterial sequences and one of the probes is non-specific for gram-positive or gram-negative bacterial sequences.

47. The kit of claim 46, wherein two of the four probes hybridize to sequence corresponding to one of the two regions of sequence encoding bacterial rRNA and the two of the four probes hybridize to sequence corresponding to the other of the two regions of sequence encoding bacterial rRNA.

48-58. (canceled)
